SEQUENCE LISTING (16 sequences)

SEQ ID NO: 1 - DNA, 3888 bp, Clostridium botulinum
  source 1..3888: mol_type = genomic DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3888 bp, synthetic construct
  misc_feature 1..3888: note = Modified BoNT/A "Cat-A"
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 4 - protein, 1296 aa, synthetic construct
  REGION 1..1296: note = Modified BoNT/A "Cat-A"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 5 - DNA, 3888 bp, synthetic construct
  misc_feature 1..3888: note = Modified BoNT/A "Cat-B"
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaagaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 6 - protein, 1296 aa, synthetic construct
  REGION 1..1296: note = Modified BoNT/A "Cat-B"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFKKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 7 - DNA, 3888 bp, synthetic construct
  misc_feature 1..3888: note = Modified BoNT/A "Cat-C"
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaagaagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 1296 aa, synthetic construct
  REGION 1..1296: note = Modified BoNT/A "Cat-C"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLKKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 9 - DNA, 3888 bp, synthetic construct
  misc_feature 1..3888: note = Modified BoNT/A "Cat-D"
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagcgtaaagagtaccgtctggcgaccaacgcttcgcaggcgggtgttgagaaaattctgagcgcgttggagatccctcgtgtccgtcgtctgagccaagtcgtggttatgaagagcaagaacgaccagggtatcactaacaagtgcaagatgaacctgcaagaccgtcgtggtaacgacatcggctttattggtttccaccagttcaacaatattgctaaactggtagcgagcaattggtacaatcgtcagattgagcgccgtagccgtcgtttgggctgtagctgggagtttatcccggtcgatgatggttggggcgaacgtccgctg

SEQ ID NO: 10 - protein, 1296 aa, synthetic construct
  REGION 1..1296: note = Modified BoNT/A "Cat-D"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKRKEYRLATNASQAGVEKILSALEIPRVRRLSQVVVMKSKNDQGITNKCKMNLQDRRGNDIGFIGFHQFNNIAKLVASNWYNRQIERRSRRLGCSWEFIPVDDGWGERPL

SEQ ID NO: 11 - protein, 1315 aa, synthetic construct
  REGION 1..1315: note = Modified BoNT/A "Chimera 1"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 12 - protein, 1327 aa, synthetic construct
  REGION 1..1327: note = Modified BoNT/A "Chimera 2"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 13 - protein, 1314 aa, synthetic construct
  REGION 1..1314: note = Modified BoNT/A "Chimera 3A"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 14 - protein, 1304 aa, synthetic construct
  REGION 1..1304: note = Modified BoNT/A "Chimera 3B"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 15 - protein, 1304 aa, synthetic construct
  REGION 1..1304: note = Modified BoNT/A "Chimera 3C"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 16 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

(19) United States
(12) Patent Application Publication
(10) Pub. No.: US 2026/0224673 A1
(43) Pub. Date: Aug. 6, 2026

(54) TREATMENT OF LIMB SPASTICITY

(71) Applicant: IPSEN BIOPHARM LIMITED, Wrexham (GB)

(72) Inventors: Nicolae GRIGORE, Wrexham (GB); Katie RUFFLE, Wrexham (GB); Philippe PICAUT, Wrexham (GB)

(21) Appl. No.: 19/351,105

(22) Filed: Oct. 6, 2025

Related U.S. Application Data
(63) Continuation of application No. 17/911,538, filed on Mar. 16, 2021, now abandoned.

(30) Foreign Application Priority Data
Mar. 16, 2020 (GB) 2003803.0
Mar. 17, 2020 (GB) 2003867.5

(51) Int. Cl. A61K 38/48; A61K 9/00; A61P 21/02

(52) U.S. Cl. CPC A61K 38/4893; A61K 9/0019; A61P 21/02; C12Y 304/24069

(57) ABSTRACT

The present invention relates to a modified botulinum neurotoxin A (BoNT/A) for use in treating limb spasticity, wherein the modified BoNT/A is administered by intramuscular injection to treat a plurality of affected muscles in a subject. Also provided are corresponding methods of treatment and uses, as well as unit dosage forms, and kits.

### CROSS-REFERENCE TO RELATED APPLICATIONS

This application is a Continuation application of U.S. application Ser. No. 17/911,538, filed Sep. 14, 2022, which is a National Stage Entry of International Application No. PCT/GB2021/050647, filed Mar. 16, 2021, which claims priority to United Kingdom Application No. 2003803.0, filed Mar. 16, 2020, and Application No. 2003867.5, filed Mar. 17, 2020, the disclosures of which are hereby incorporated by reference in their entireties.

### INCORPORATION BY REFERENCE OF SEQUENCE LISTING

The instant application contains a Sequence Listing which has been submitted electronically in XML format and is hereby incorporated by reference in its entirety. Said XML copy, created on Sep. 30, 2025, is named IPS-017US2_SL.xml and is 53,709 bytes in size.

### DETAILED DESCRIPTION

The present invention relates to treatment of limb spasticity using a modified botulinum neurotoxin A (BoNT/A).

### BACKGROUND

Spasticity is a motor symptom characterised by an increase in velocity dependent stretch reflexes, with exaggerated tendon jerks, resulting from hyperexcitability of the stretch reflex, as one component of the motor neuron syndrome. Spasticity is associated with various neurological disorders e.g. multiple sclerosis, cerebral or spinal cord injuries, traumatic brain injury (TBI), and cerebrovascular disorders (stroke). It is characterised by motor impairment (increased muscle tone, abnormal limb posture, excessive contraction of antagonist muscles and hyperactive reflexes) functional disability, pain, and discomfort.

Adult upper limb (AUL) spasticity is a common complication after stroke; it is often painful (associated with spastic muscle contraction) and can cause significant disability by interfering with upper limb movement and limiting use of the limb for active functional tasks. In severe cases, it can also impede ‘passive function’, such as washing, dressing and caring for the affected limb, thereby increasing the burden on caregivers. Secondary complications may include poor self-esteem and body image, impaired quality of life (reduced social and family interaction) and pressure ulcers.

Upper limb post-stroke spasticity management is challenging due to the diversity of patient presentation and their goals. In these patients, muscle hypertonia typically manifests in several common clinical patterns determined by the muscles affected, which is in turn related to the size, location and age of the Central Nervous System lesions. These patterns were described in an international, cross-sectional survey of clinicians in 31 countries and subsequently were used to develop a classification of 5 typical patterns for AUL spasticity. These are defined with respect to the position of the shoulder, elbow, forearm, and wrist joints. Current clinical consensus and existing guidelines recommend that the exact pattern of spasticity should be considered when selecting the treatment for patients and botulinum neurotoxin A (BoNT/A) therapy should be tailored accordingly with the appropriate muscles selected for injection. However, no two patients are identical and there is a need for custom/personalised treatment regimens.

An expert panel looked further on these patterns in order to provide guidance of treatment parameters for upper limb spasticity (ULS) and the targeted muscles for each of the clinical patterns (see FIG. 1). Significantly the panel defined three aggregate posture combinations to target the treatment with BoNT. These were:
(1) Adducted shoulder, Flexed elbow, Pronated forearm, Flexed wrist, Clenched fist;
(2) Flexed elbow, Pronated forearm, Flexed wrist, Clenched fist; and
(3) Flexed wrist, Clenched fist.

The four upper limb joints shown in FIG. 1 are known to be involved in >90% of subjects (see FIG. 1). The majority of spastic upper limb patterns (four out of five) result from internal rotation and adduction of the shoulder and flexion at the elbow joint, with differences in posture resulting from the posture of the forearm and the wrist.

Overall ULS treatment is aimed at relieving the signs and symptoms of spasticity-reducing muscle spasms and pain, improving posture, facilitating mobility and dexterity (voluntary motor functions reaching for, grasping, moving and releasing), minimising contractures and deformity and improving patient ease of care as well as hygiene/self-care and/or quality of life. Available pharmacological treatment options like oral (benzodiazepines, baclofen, tizanidine and dantrolene) and intrathecal (baclofen) medications mostly cause nonselective muscle weakness with side effects such as generalised weakness and adverse effects on the central nervous system, including ataxia, drowsiness, sedation and even withdrawal symptoms.

The most effective approach for the treatment of spasticity is a combination of physiotherapy and intramuscular (i.m.) injections of BoNT/A that has been recommended by several guidelines in the recent years. BoNT/A has emerged as a treatment of choice as it has minimal systemic side effects, and being a locally injectable treatment, can be adapted to the individual's disease presentation by selectively targeting the affected muscles.

Dysport® is a medicinal product containing drug substance BoNT/A haemagglutinin complex (BTX-A-HAC) isolated and purified from Clostridium botulinum type A strain. Several other medicinal BoNT/A products naturally produced by Clostridium botulinum are also on the market (e.g. BOTOX® and XEOMIN®).

BoNT/A selectively inhibits the release of acetylcholine from the presynaptic nerve terminals and thus blocks cholinergic transmission at the neuromuscular junction inducing a reduction in the muscle contraction and muscle tone, causing the injected muscles to relax. This mechanism of action has been used therapeutically to treat several clinical neurological conditions including focal dystonias, focal muscle spasticity and aesthetic conditions for more than two decades.

However, the duration of action of the currently available BoNT/A products is about 12 to 14 weeks, which is when the new nerve endings sprout allowing the nerve function to return to normal, and the original symptoms reappear. Consequently, for the effect to be maintained, injections need to be repeated periodically.

Thus, the frequency of BoNT/A injections is an important consideration for the treatment of spasticity, considering the chronicity of the condition and long-term nature of the treatment required. Indeed, it has an impact on the direct and indirect health costs involved for the patients and caregivers, the logistics for injections within the hospitals/clinics, and most importantly the quality of life of patients.

Dysport® is approved for the treatment of upper and lower limb spasticity with a maximum total dose per treatment session of 1,500 Units (see FIGS. 2—1,000 Units maximum for treating upper limb spasticity). A clinician is required to administer Dysport® to a plurality of muscles on the limbs up to the upper threshold of 1,500 Units total per treatment session. The clinician is forced to make difficult choices during treatment of a patient. In other words, in conventional treatment regimens, a clinician must find a balance between the relatively low total amount of BoNT/A that can be administered (1,500 Units or 1,000 Units for upper limb spasticity—necessitated by the highly toxic nature of BoNT/A) and the effective amount at a plurality of different muscles. Hence, certain muscles are neglected while others receive a suboptimal amount of BoNT/A, resulting in suboptimal therapy. Furthermore, the current treatment regimens exclude treatment of the shoulder, which requires multiple unit doses at a plurality of muscles thereof.

Moreover, the conventional treatment regimens are complicated and result in clinicians under-dosing in an effort to avoid toxicity to the patient. There is thus a need for a convenient, safe, and effective single dose unit and a corresponding guide to the number of units that can be administered to a limb (including the number of injection sites per muscle) in a treatment session without resultant patient toxicity.

In conclusion, there is a need for an improved treatment for limb spasticity that would allow an individualised patient-centric approach to tailor the treatment according to the targeted clinical pattern permitting different combinations of limb muscles to be injected depending on the distribution, extent and severity of spasticity, while avoiding toxicity and providing a longer-lasting treatment (resulting in less frequent administration). There is also a need for an improved treatment regimen that allows treatment of neglected muscles, such as those of the shoulder.

The present invention overcomes one or more of the above-mentioned problems.

### SUMMARY OF THE INVENTION

The present inventors have surprisingly found that a modified BoNT/A finds particular utility in treating limb spasticity. The modified BoNT/A may comprise one or more modifications of surface exposed amino acid residues resulting in an increased net positive charge. The increased charge promotes electrostatic interactions between the polypeptide and anionic extracellular components, thereby promoting binding between the polypeptide and cell surface. In turn this increases retention at (reduces diffusion away from) a site of administration and results in an increased duration of action (e.g. 6-9 months). Alternatively, a modified BoNT/A may comprise a BoNT/A light-chain and translocation domain and a BoNT/B receptor binding domain (H_{C} domain), which similarly results in a modified BoNT/A that exhibits increased retention at (reduced diffusion away from) a site of administration and increased duration of action (e.g. 6-9 months). Advantageously, modified BoNT/A has a safety profile that is improved when compared to unmodified BoNT/A (e.g. Dysport®). This improved safety profile may be expressed by the high Safety Ratio described herein for the modified BoNT/A.

Based on the pre-clinical data herein (see Example 6) it has been shown that a higher total amount of modified BoNT/A can be administered to a subject while achieving a similar safety profile to unmodified BoNT/A (e.g. Dysport®) while at such high doses. Thus, modified BoNT/A can be injected at a greater number of muscles/sites in the treatment of limb spasticity before reaching the maximum total dose. This is a significant and advantageous finding and yields an improved treatment of limb spasticity while providing clinicians with a greater range of treatment options. For the first time, it also provides the option of being able to treat additional large muscles such as those of the shoulder, while also treating the elbow, forearm, and/or wrist well within the maximum dose. The treatment may be improved in that it provides for longer-lasting treatment (resulting in less frequent administration) and/or is capable of being tailored for the subject and/or results in an improved quality of life of a subject when compared to treatment with unmodified BoNT/A (e.g. Dysport®). The treatment of the invention is improved compared to conventional treatment regimens.

Moreover, the present invention provides a convenient, safe, and effective single unit dose as well as a total (maximum) dosage that can safely be administered in a single treatment. The present invention also provides a corresponding guide to the number of times at which said unit dose can be administered to a limb (e.g. including the number of injection sites per muscle) without resultant patient toxicity. Treatment in accordance with the present invention is thus much less complicated for the clinician and helps avoid under-dosing and/or over-dosing.

### DETAILED DESCRIPTION

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 53 Units to 948 Units of modified BoNT/A (preferably by way of a unit dose of 53 Units to 948 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 14,220 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In a related aspect the invention provides a method for treating limb spasticity, the method comprising administering a modified botulinum neurotoxin A (BoNT/A) by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of 31 Units to 707 Units of modified BoNT/A (preferably by way of a unit dose of 31 Units to 707 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 10,605 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 26.5 Units to 474 Units of modified BoNT/A (preferably by way of a unit dose of 26.5 Units to 474 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 7,110 Units, and
wherein the modified BoNT/A comprises a BoNT/A light-chain and translocation domain, and a BoNT/B receptor binding domain (H_{C} domain).

In one aspect the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 15.5 Units to 353.5 Units of modified BoNT/A (preferably by way of a unit dose of 15.5 Units to 353.5 Units of modified BoNT/A per injection site) at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 5302.5 Units, and
wherein the modified BoNT/A comprises a BoNT/A light-chain and translocation domain, and a BoNT/B receptor binding domain (H_{C} domain).

An “affected muscle” is a muscle exhibiting a symptom of spasticity or contributing to the spasticity of an affected limb. For example, said muscle may exhibit increased muscle tone or stiffness.

The plurality of affected muscles are selected from a first and second group as described herein. The plurality of affected muscles selected may be at least one muscle of the first group and/or at least one muscle of the second group. Alternatively, a plurality of affected muscles may be two or more muscles from the same group (e.g. two or more first group muscles or two or more second group muscles). Preferably, the plurality of affected muscles include at least one first group muscle and at least one second group muscle.

The plurality of muscles may be muscles of the same limb or muscles of different limbs. It is preferred, however, that the plurality of muscles are muscles of the same limb. The present invention encompasses treating spasticity in one or more limbs simultaneously. For example, a modified BoNT/A may be administered to one or both upper limbs of a subject per treatment session, one or both lower limbs per treatment session, or a combination of lower and upper limbs per treatment session. Regardless of whether two or more limbs are treated, it is preferred that at least 2 muscles are treated per limb, e.g. at least 3, 4 or 5 muscles per limb.

A modified BoNT/A for use in the present invention may be either a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue or a modified BoNT/A comprising a BoNT/A light-chain and translocation domain, and a BoNT/B receptor binding domain (H_{C} domain).

Preferably, a modified BoNT/A for use in the present invention is a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue.

Potency of a modified BoNT/A for use according to the invention is preferably determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice. An amount of a modified BoNT/A that corresponds to 1 Unit in said assay may be at least 1 pg, 2 pg, 3 pg, 4 pg, 5 pg, 6 pg, 7 pg, 8 pg or 9 pg. An amount of a modified BoNT/A that corresponds to 1 Unit in said assay may be ≤45 pg, ≤40 pg, ≤30 pg, ≤25 pg, ≤20 pg, ≤19 pg, ≤18 pg, ≤17 pg, ≤16 pg, ≤15 pg, ≤14 pg, ≤13 pg, ≤12 pg, ≤11 pg, ≤10 pg, ≤9 pg, ≤8 pg, ≤7 pg or ≤6 pg.

Where a modified BoNT/A for use in the invention is a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue, an amount of a modified BoNT/A that corresponds to 1 Unit in said assay may be 1-15 pg, such as 5-10 pg. Preferably, an amount of a modified BoNT/A that corresponds to 1 Unit in said assay may be 8-9 pg, more preferably 8.4 pg.

Where a modified BoNT/A for use in the invention is modified BoNT/A comprising a BoNT/A light-chain and translocation domain, and a BoNT/B receptor binding domain (H_{C} domain), an amount of a modified BoNT/A that corresponds to 1 Unit in said assay may be 15-35 pg, such as 20-30 pg. Preferably, an amount of a modified BoNT/A that corresponds to 1 Unit in said assay may be 23-25 pg, more preferably 24.0 pg.

The term “up to” when used in reference to a value (e.g. up to 14,220 Units) means up to and including the value recited. Thus, as an example, reference to administering “up to 14,220 Units” of modified BoNT/A encompasses administration of 14,220 Units of modified BoNT/A as well as administration of less than 14,220 Units of modified BoNT/A.

Preferably, a dose of modified BoNT/A is administered by intramuscular injection at an affected muscle. More preferably, a single unit dose is administered per injection site. The term “a single unit dose is administered” means substantially all of a single unit dose is administered. For example, a residual amount (e.g. up to 1%, 0.1% or 0.01%) of the unit dose may remain in a vial in which the modified BoNT/A has been reconstituted. However, preferably all of a single unit dose is administered (e.g. at one or more injection sites). Depending on the nature of the muscle, either a single unit dose is administered (i.e. to a muscle selected from a first group described herein) or multiple unit doses are administered (i.e. to a muscle selected from a second group described herein). Said single unit dose or multiple unit doses may be administered at one or more injection sites (e.g. per muscle). For example, in some embodiments, less than a single unit dose may be administered per injection site. In a preferred embodiment, some muscles are injected at one site only (i.e. muscles selected from a first group described herein) and some muscles are injected at two or more sites (i.e. muscles selected from a second group described herein).

In one embodiment, a single unit dose is administered at a plurality of injection sites at an affected first group muscle and/or multiple unit doses are administered at a plurality of injection sites at an affected second group muscle.

The unit dose can be expressed in terms of Units of modified BoNT/A.

The unit dose may be 53 Units to 948 Units of modified BoNT/A. An upper limit of the unit dose range may be 925, 900, 850, 800, 750, 700, 650, 600, 550, 500, 450, 400, 350, 300, 250, 200, 150 or 100 Units of modified BoNT/A, preferably the upper limit is 889 Units. A lower limit of the unit dose range may be 55, 60, 65, 70, 75, 80, 85, 90, 100, 150, 200, 250, 300, 350, 400, 450, 500, 550, 600, 650, 700, 750, 800, 850 or 900 Units of modified BoNT/A, preferably the lower limit is 59 Units. Preferably, the unit dose of modified BoNT/A is 59 Units to 889 Units of modified BoNT/A, for example 200 Units to 600 Units.

Most preferably a unit dose of modified BoNT/A is 237 to 355 Units, such as 284 to 308 Units.

The unit dose may be 31 Units to 707 Units of modified BoNT/A. An upper limit of the unit dose range may be 700, 650, 600, 550, 500, 450, 400, 350, 300, 250, 200, 150 or 100 Units of modified BoNT/A, preferably the upper limit is 666 Units. A lower limit of the unit dose range may be 40, 45, 50, 60, 65, 70, 75, 80, 85, 90, 100, 150, 200, 250, 300, 350, 400, 450, 500, 550, 600, 650, or 700 Units of modified BoNT/A, preferably the lower limit is 42 Units. Preferably, the unit dose of modified BoNT/A is 42 Units to 666 Units of modified BoNT/A, for example 200 Units to 400 Units. These unit doses may be particularly relevant when the modified BoNT/A comprises a BoNT/A light-chain and translocation domain, and a BoNT/B receptor binding domain (H_{C} domain)

Alternatively or additionally, a unit dose may be expressed in terms of an amount of modified BONT/A. Thus, in one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 450 pg to 8,000 pg of modified BoNT/A (preferably by way of a unit dose of 450 pg to 8,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 120,000 pg, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another aspect, the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 450 pg to 8,000 pg of modified BoNT/A (preferably by way of a unit dose of 450 pg to 8,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 120,000 pg, and
wherein the modified BoNT/A comprises a BoNT/A light-chain and translocation domain, and a BoNT/B receptor binding domain (H_{C} domain).

In another aspect, the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 750 pg to 17,000 pg of modified BoNT/A (preferably by way of a unit dose of 750 pg to 17,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 255,000 pg, and
wherein the modified BoNT/A comprises a BoNT/A light-chain and translocation domain, and a BoNT/B receptor binding domain (H_{C} domain).

Corresponding uses (in the manufacture of a medicament) and methods of treatment are also provided.

In one aspect the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 450 pg to 8,000 pg of modified BoNT/A (preferably by way of a unit dose of 450 pg to 8,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 120,000 pg, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another aspect, the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 450 pg to 8,000 pg of modified BoNT/A (preferably by way of a unit dose of 450 pg to 8,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 120,000 pg, and
wherein the modified BoNT/A comprises a BoNT/A light-chain and translocation domain, and a BoNT/B receptor binding domain (H_{C} domain).

In another aspect, the invention provides a modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of 750 pg to 17,000 pg of modified BoNT/A (preferably by way of a unit dose of 750 pg to 17,000 pg of modified BoNT/A per injection site) at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, and the flexor hallucis longus; and
   a second group comprising: the triceps brachii (long head), the subscapularis, the pectoralis major, the latissimus dorsi, the biceps brachii, the brachialis, the soleus, and the tibialis posterior; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 255,000 pg, and
wherein the modified BoNT/A comprises a BoNT/A light-chain and translocation domain, and a BoNT/B receptor binding domain (H_{C} domain).

Corresponding uses (in the manufacture of a medicament) and methods of treatment are also provided.

The unit dose may be 450 pg to 8,000 pg of modified BoNT/A. An upper limit of the unit dose range may be 7,750, 7,500, 7,000, 6,000, 5,000, 4,000, 3,000, 2,000 or 1,000, pg of modified BoNT/A, preferably the upper limit is 7,500 pg. A lower limit of the unit dose range may be 475, 500, 600, 700, 800, 900, 1,000, 1,500, 2,000, 3,000, 4,000, 5,000, 6,000 or 7,000 pg of modified BoNT/A, preferably the lower limit is 500 pg. Preferably, the unit dose of modified BoNT/A is 500 pg to 7,500 pg of modified BoNT/A, e.g. 4,000 pg to 6,000 pg.

Most preferably a unit dose of modified BoNT/A is 2,000 to 3,000 pg, such as 2,400 to 2,600 pg.

The unit dose may be 750 pg to 17,000 pg of modified BoNT/A. An upper limit of the unit dose range may be 16,500, 15,500, 14,500, 13,500, 12,500, 11,500, 10,500, 9,500, 8,500, 7,500, 6,500, 5,500, 4,500, 3,500, 2,500, 1,500 or 500 pg of modified BoNT/A, preferably the upper limit is 16,000 pg. A lower limit of the unit dose range may be 750, 850, 950, 1000, 1500, 2000, 2,500, 3,000, 3,500, 4,000, 4,500 or 5,000 pg of modified BoNT/A, preferably the lower limit is 1000 pg. Preferably, the unit dose of modified BoNT/A is 1000 pg to 16,000 pg of modified BoNT/A, e.g. 4,000 pg to 6,000 pg. These unit doses may be particularly relevant when the modified BoNT/A comprises a BoNT/A light-chain and translocation domain, and a BoNT/B receptor binding domain (H_{C} domain).

A unit dose of modified BoNT/A may also be expressed in both Units and amounts (pg) simultaneously.

A single unit dose of modified BoNT/A is administered to a muscle selected from the first group described herein when said first group muscle is selected for treatment.

Multiple unit doses of modified BoNT/A are administered to a muscle selected from the second group. For example, at least 2×, 3× or 4× unit doses may be administered. In some embodiments 2-4× unit doses may be administered. Preferably, 2× unit doses of modified BoNT/A are administered to a muscle selected from the second group.

Some muscles can be present in the first muscle group and second muscle group. Thus, a clinician can decide whether to administer a single unit dose to said muscle or multiple unit doses.

The limb spasticity may be upper limb spasticity or lower limb spasticity.

When treating lower limb spasticity, modified BoNT/A may be administered to a plurality of muscles selected from: a first group comprising (preferably consisting of): the gastrocnemius medial head, the gastrocnemius lateral head, the gastrocnemius, the flexor digitorum longus, the flexor hallucis longus, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and a second group comprising (preferably consisting of): the soleus, the tibialis posterior, the iliopsoas, and the gastrocnemius.

When administering to the flexor hallucis longus, in some instances a single unit dose may be administered, while in other instances 2× the unit dose may be administered.

Suitable dosages by lower limb muscle are shown below:

| Muscles Injected | Dosage |
|---|---|
| Gastrocnemius (Medial head) | 1x UD |
| Gastrocnemius (Lateral head) | 1x UD |
| Soleus | 2x UD or 3x UD |
| Tibialis posterior | 2x UD |
| Flexor digitorum longus (FDL) | 1x UD |
| Flexor hallucis longus | 1x or 2x UD |

The modified BoNT/A may be administered to one or more of the muscles indicated at the dosages indicated in the table above.

Modified BoNT/A may be administered to one or more of the following muscles as follows at the following dosages:

| Muscles Injected | Dosage |
|---|---|
| Adductor magnus | 1x UD |
| Adductor longus | 1x UD |
| Adductor brevis | 1x UD |
| Gracilis | 1x UD |
| Medial hamstrings | 1x UD |
| Lateral hamstrings | 1x UD |
| Tensor fascia lata | 1x UD |
| Rectus femoris | 1x UD |
| Vastus lateralis | 1x UD |
| Vastus medialis | 1x UD |
| Vastus intermedius | 1x UD |
| Gluteus maximus | 1x UD |
| Tibialis anterior | 1x UD |
| FDL | 1x UD |
| Flexor digitorum brevis (FDB) | 1x UD |
| Flexor hallucis longus | 1x UD |
| Extensor hallucis longus | 1x UD |
| Flexor hallucis brevis | 1x UD |
| Iliopsoas | 2x UD |
| Gastrocnemius | 2x UD |
| Tibialis posterior | 2x UD |
| Soleus | 2x UD |

Preferably, the limb spasticity is upper limb spasticity.

When treating upper limb spasticity, modified BoNT/A may be administered to a plurality of affected muscles selected from: a first group comprising (preferably consisting of): the flexor digitorum superficialis, the flexor digitorum profundus, the flexor carpi radialis, the flexor carpi ulnaris, the brachioradialis, the pronator teres, the deltoid, the levator scapulae, the pronator quadratus, the flexor policis longus, the adductor policis, the flexor policis brevis, the palmaris longus, the lumbricales, and the opponens policis; and a second group comprising (preferably consisting of): the triceps brachii (long head), the subscapularis, the pectoralis (e.g. the pectoralis major), the latissimus dorsi, the biceps brachii, the brachialis, the brachioradialis, and the teres major.

Preferably, when treating upper limb spasticity, modified BoNT/A may be administered to a plurality of affected muscles selected from: a first group comprising (preferably consisting of): the flexor digitorum superficialis, the flexor digitorum profundus, the flexor carpi radialis, the flexor carpi ulnaris, the brachioradialis, the pronator teres, and the biceps brachii; and a second group comprising (preferably consisting of): the triceps brachii (long head), the subscapularis, the pectoralis major, the latissimus dorsi, the biceps brachii, and the brachialis.

More preferably, modified BoNT/A may be administered to a muscle associated with shoulder spasticity, such as adduced shoulder. Modified BoNT/A may be administered to at least one of the latissimus dorsi, the subscapularis, the pectoralis major, and the triceps brachii (long head). For example, modified BoNT/A may be administered to at least two or three (preferably all) of the latissimus dorsi, the subscapularis, the pectoralis major or the triceps brachii (long head).

When administering to the biceps brachii in some instances a single unit dose may be administered, while in other instances a 2× unit dose may be administered. Thus, the biceps brachii fall into both a first and second group of muscles. Preferably, 2× the unit dose is administered to the biceps brachii and this muscle is included in a second group of muscles.

Suitable dosages by upper limb muscle are shown below:

| Muscles Injected | Dosage |
|---|---|
| Flexor Digitorum Superficialis | 1x UD |
| (FDS) | |
| Flexor Digitorum Profundus | 1x UD |
| (FDP) | |
| Flexor Carpi Radialis (FCR) | 1x UD |
| Flexor Carpi Ulnaris (FCU) | 1x UD |
| Brachioradialis | 1x UD |
| Brachialis | 2x UD |
| Pronator Teres | 1x UD |
| Biceps Brachii | 2x UD |
| | 1x or 2x UD |
| Triceps Brachii (long head) | 2x UD |
| Pectoralis Major | 2x UD |
| Subscapularis | 2x UD |
| Latissimus Dorsi | 2x UD |

The modified BoNT/A may be administered to one or more of the muscles indicated at the dosages indicated in the table above.

Modified BoNT/A may be administered to one or more of the following muscles as follows at the following dosages:

| Muscles Injected | Dosage |
|---|---|
| Deltoid | 1x UD |
| Levator scapulae | 1x UD |
| Pronator Quadratus | 1x UD |
| Pronator Teres | 1x UD |
| Brachioradialis | 1x UD |
| FCR | 1x UD |
| FCU | 1x UD |
| FDS | 1x UD |
| FDP | 1x UD |
| Flexor policis longus | 1x UD |
| Adductor policis | 1x UD |
| Flexor policis brevis | 1x UD |
| Palmaris longus | 1x UD |
| Lumbricales | 1x UD |
| Opponens policis | 1x UD |
| Latissimus Dorsi | 2x UD |
| Pectoralis | 2x UD |
| Brachioradialis | 2x UD |
| Teres Major | 2x UD |
| Subscapularis | 2x UD |
| Brachialis | 2x UD |
| Biceps Brachii | 2x UD |

The total number of unit doses administered in a given treatment may be up to 15× the unit dose. In other words, in one embodiment, 15× single unit doses may be administered at 15× injections sites. In another embodiment, 15× single unit doses may be administered at more than 15× injections sites. The total number of unit doses will be divided according to the muscles treated, e.g. 2× unit doses may be administered to the latissimus dorsi, 2× to the subscapularis, 2× to the pectoralis major, and 1× to the flexor carpi ulnaris yielding a total multiple of unit doses administered of 7×. For example, the total number of unit doses administered may be up to 14×, 13×, 12×, 11×, 10×, 9×, 8× or 7×. The total number of unit doses administered may be at least 2×, 3×, 4×, 5×, 6×, 7× the unit dose, preferably at least 2×. The total number of unit doses administered may be 2× to 15×, 7× to 15× or 10× to 14×. Preferably, the number of unit doses administered is 15×.

A total dose administered when carrying out the treatment regimen of the present invention may be up to 14,220 Units. In other words, the total amount of modified BoNT/A administered at a given treatment session may be up to 14,220 Units. The total dose may be up to 14,000, 13,000, 12,000, 11,000, 10,000, 9,000, 8,000, 7,000, 6,000, 5,000, 4,000, 3,000, 2,000 or 1,000 Units. Preferably, the total dose may be up to 13,335 Units of modified BoNT/A. The total dose may be at least 106, 150, 200, 250, 300, 350, 400, 450, 500, 550, 600, 650, 700, 750, 800, 850, 900, 950, 1,000, 1,500, 2,000, 3,000, 4,000, 5,000, 6,000, 7,000, 8,000, 9,000, 10,000, 11,000, 12,000 or 13,000 Units. Preferably, the total dose may be at least 106 Units, more preferably at least 118 Units of modified BoNT/A, e.g. at least 795 units. The total dose may be 118-13,335 Units, preferably 795-13,335 Units. More preferably, the total dose administered is 9,000-13,335 Units.

A total dose administered when carrying out the treatment regimen of the present invention may be up to 120,000 pg. In other words, the total amount of modified BoNT/A administered at a given treatment session may be up to 120,000 pg. The total dose may be up to 115,000, 110,000, 100,000, 90,000, 80,000, 70,000, 60,000, 50,000, 40,000, 30,000, 20,000, 10,000 or 5,000 pg. Preferably, the total dose may be up to 112,500 pg of modified BoNT/A. The total dose may be at least 900, 1,000, 2,000, 3,000, 4,000, 5,000, 7,500, 10,000, 12,500, 15,000, 20,000, 30,000, 40,000, 50,000, 60,000, 70,000, 80,000, 90,000 or 100,000 pg. Preferably, the total dose may be at least 900 pg, more preferably at least 1,000 pg of modified BoNT/A, e.g. at least 6,000 pg. The total dose may be 1,000-112,500 pg, preferably 60,000-112,500 pg. More preferably, the total dose administered is 7,500-112,500 pg.

A total dose administered when carrying out the treatment regimen of the present invention may be up to 10,605 Units. In other words, the total amount of modified BoNT/A administered at a given treatment session may be up to 10,605 Units. The total dose may be up to 10,000, 9,000, 8,000, 7,000, 6,000, 5,000, 4,000, 3,000, 2,000 or 1,000 Units. Preferably, the total dose may be up to 9,990 Units of modified BoNT/A. The total dose may be at least 50, 100, 150, 200, 250, 300, 350, 400, 450, 500, 550, 600, 650, 700, 750, 800, 850, 900, 950, 1,000, 1,500, 2,000, 3,000, 4,000, 5,000, 6,000, 7,000, 8,000, or 9,000. Preferably, the total dose may be at least 62 Units, more preferably at least 84 Units of modified BoNT/A, e.g. at least 465 units. The total dose may be 84-9,990 Units, preferably 465-9,990 Units. More preferably, the total dose administered is 630-9,990 Units. These total dosages may be particularly relevant where the modified BONT comprises a BoNT/A light-chain and translocation domain, and a BoNT/B receptor binding domain (H_{C} domain).

A total dose administered when carrying out the treatment regimen of the present invention may be up to 255,000 pg. In other words, the total amount of modified BoNT/A administered at a given treatment session may be up to 255,000 pg. The total dose may be up to 240,000, 220,000, 200,000, 180,000, 160,000, 140,000,110,000, 100,000, 90,000, 80,000, 70,000, 60,000, 50,000, 40,000, 30,000, 20,000, 10,000 or 5,000 pg. Preferably, the total dose may be up to 240,000 pg of modified BoNT/A. The total dose may be at least 900, 1,000, 2,000, 3,000, 4,000, 5,000, 7,500, 10,000, 12,500, 15,000, 20,000, 30,000, 40,000, 50,000, 60,000, 70,000, 80,000, 90,000, 100,000, 120,000, 150,000, 175,000, 200,000 or 220,000 pg. Preferably, the total dose may be at least 1500 pg, more preferably at least 2000 pg of modified BoNT/A, e.g. at least 12,000 pg. The total dose may be 2000-240,000 pg, preferably 128,000-240,000 pg. More preferably, the total dose administered is 15,000-240,000 pg. These total dosages may be particularly relevant where the modified BONT comprises a BoNT/A light-chain and translocation domain, and a BoNT/B receptor binding domain (H_{C} domain).

The skilled person will take into consideration when a subject has recently had (or is subsequently having) additional treatment with a clostridial neurotoxin (e.g. BoNT), e.g. as part of a cosmetic treatment or treatment for a different indication. Using techniques routine in the art, the skilled person will adapt the present treatment regimen accordingly.

It is preferred that the limb spasticity for treatment in accordance with the present invention is adult limb spasticity. Preferably, the dosage details provided above are for treating adult limb spasticity. However, the treatment of paediatric limb spasticity is also encompassed. When treating paediatric limb spasticity, the combined total dosage is typically 50% or less than that used when treating adult limb spasticity. In some embodiments, the combined total dosage is typically 70% or less (e.g. 67% or less) than that used when treating adult limb spasticity.

A suitable unit dose for treating paediatric limb spasticity may be 26.5 Units to 474 Units of modified BoNT/A and the total dose administered during the treatment is up to 7,110 Units. An upper limit of the unit dose range for treating paediatric limb spasticity may be 400, 350, 300, 250, 200, 150, 100 or 50 Units of modified BoNT/A, preferably the upper limit is 444.5 Units. A lower limit of the unit dose range for treating paediatric limb spasticity may be 30, 35, 40, 45, 50, 55, 60, 65, 70, 75, 80, 85, 90, 100, 150, 200, 250, 300, 350 or 400 Units of modified BoNT/A, preferably the lower limit is 29.5 Units. Preferably, the unit dose of modified BoNT/A for treating paediatric limb spasticity is 29.5 Units to 474 Units (more preferably 29.5 Units to 444.5 Units) of modified BoNT/A, for example 100 Units to 300 Units.

A suitable unit dose for treating paediatric limb spasticity may be 37.1 Units to 663.6 Units of modified BoNT/A and the total dose administered during the treatment is up to 9,954 Units. An upper limit of the unit dose range for treating paediatric limb spasticity may be 600, 550, 500, 450, 400, 350, 300, 250, 200, 150, 100 or 50 Units of modified BoNT/A, preferably the upper limit is 622.3 Units. A lower limit of the unit dose range for treating paediatric limb spasticity may be 40, 45, 50, 55, 60, 65, 70, 75, 80, 85, 90, 95, 100, 150, 200, 250, 300, 350, 400 or 450 Units of modified BoNT/A, preferably the lower limit is 41.3 Units. Preferably, the unit dose of modified BoNT/A for treating paediatric limb spasticity is 41.3 Units to 622.3 Units of modified BoNT/A, for example 100 Units to 700 Units.

A suitable unit dose for treating paediatric limb spasticity may be 35.1 Units to 635.16 Units of modified BoNT/A and the total dose administered during the treatment is up to 9,527.4 Units. An upper limit of the unit dose range for treating paediatric limb spasticity may be 600, 550, 500, 450, 400, 350, 300, 250, 200, 150, 100 or 50 Units of modified BoNT/A, preferably the upper limit is 595.63 Units. A lower limit of the unit dose range for treating paediatric limb spasticity may be 40, 45, 50, 55, 60, 65, 70, 75, 80, 85, 90, 95, 100, 150, 200, 250, 300, 350, 400 or 450 Units of modified BoNT/A, preferably the lower limit is 39.5 Units. Preferably, the unit dose of modified BoNT/A for treating paediatric limb spasticity is 39.5 Units to 635.2 Units of modified BoNT/A, for example 100 Units to 700 Units.

A suitable unit dose for treating paediatric limb spasticity may be 225 pg to 4,000 pg of modified BoNT/A and the total dose administered during the treatment is up to 60,000 pg. An upper limit of the unit dose range for treating paediatric limb spasticity may be 3,500, 3,000, 2,000, 1,000 or 500 pg of modified BoNT/A, preferably the upper limit is 3,750 pg. A lower limit of the unit dose range for treating paediatric limb spasticity may be 250, 300, 350, 400, 450, 500, 600, 700, 800, 900, 1,000, 1,500, 2,000, 3,000 or 3,500 pg of modified BoNT/A, preferably the lower limit is 250 pg. Preferably, the unit dose of modified BoNT/A for treating paediatric limb spasticity is 250 pg to 3,750 pg of modified BoNT/A, e.g. 2,000 pg to 3,000 pg.

A suitable unit dose for treating paediatric limb spasticity may be 315 pg to 5,600 pg of modified BoNT/A and the total dose administered during the treatment is up to 84,000 pg. An upper limit of the unit dose range for treating paediatric limb spasticity may be 5,000, 4,000, 3,000, 2,000, 1,000 or 500 pg of modified BoNT/A, preferably the upper limit is 5,250 pg. A lower limit of the unit dose range for treating paediatric limb spasticity may be 350, 400, 450, 500, 600, 700, 800, 900, 1,000, 1,500, 2,000, 3,000 or 3,500 pg of modified BoNT/A, preferably the lower limit is 350 pg. Preferably, the unit dose of modified BoNT/A for treating paediatric limb spasticity is 350 pg to 5,250 pg of modified BoNT/A, e.g. 2,000 pg to 3,000 pg.

A suitable unit dose for treating paediatric limb spasticity may be 301.5 pg to 5,360 pg of modified BoNT/A and the total dose administered during the treatment is up to 80,400 pg. An upper limit of the unit dose range for treating paediatric limb spasticity may be 5,000, 4,000, 3,000, 2,000, 1,000 or 500 pg of modified BoNT/A, preferably the upper limit is 5,025 pg. A lower limit of the unit dose range for treating paediatric limb spasticity may be 300, 350, 400, 450, 500, 600, 700, 800, 900, 1,000, 1,500, 2,000, 3,000 or 3,500 pg of modified BoNT/A, preferably the lower limit is 335 pg. Preferably, the unit dose of modified BoNT/A for treating paediatric limb spasticity is 335 pg to 5,025 pg of modified BoNT/A, e.g. 2,000 pg to 3,000 pg.

A total dose administered when carrying out the treatment regimen of the present invention for treating paediatric limb spasticity may be up to 7,110 Units. In other words, the total amount of modified BoNT/A administered at a given treatment session for treating paediatric limb spasticity may be up to 7,110 Units. The total dose for treating paediatric limb spasticity may be up to 7,000, 6,000, 5,000, 4,000, 3,000, 2,000, 1,000 or 500 Units. Preferably, the total dose for treating paediatric limb spasticity may be up to 6,667.5 Units of modified BoNT/A. The total dose for treating paediatric limb spasticity may be at least 53, 60, 70, 80, 90, 100, 150, 200, 250, 300, 350, 400, 450, 500, 550, 600, 650, 700, 750, 800, 850, 900, 950, 1,000, 1,500, 2,000, 3,000, 4,000, 5,000, 6,000 or 7,000 Units. Preferably, the total dose for treating paediatric limb spasticity may be at least 53 Units, more preferably at least 59 Units of modified BoNT/A, e.g. at least 397.5 units. The total dose for treating paediatric limb spasticity may be 59-6,667.5 Units, preferably 397.5-6,667.5 Units. More preferably, the total dose administered is 4,500-6,667.5 Units.

A total dose administered when carrying out the treatment regimen of the present invention for treating paediatric limb spasticity may be up to 60,000 pg. In other words, the total amount of modified BoNT/A administered at a given treatment session for treating paediatric limb spasticity may be up to 60,000 pg. The total dose for treating paediatric limb spasticity may be up to 55,000, 50,000, 40,000, 30,000, 20,000, 10,000, 5,000, 3,000 or 2,500 pg. Preferably, the total dose for treating paediatric limb spasticity may be up to 56,250 pg of modified BoNT/A. The total dose for treating paediatric limb spasticity may be at least 500, 600, 700, 800, 900, 1,000, 2,000, 3,000, 4,000, 5,000, 7,500, 10,000, 12,500, 15,000, 20,000, 30,000, 40,000, 50,000 or 55,000 pg. Preferably, the total dose for treating paediatric limb spasticity may be at least 450 pg, more preferably at least 500 pg of modified BoNT/A, e.g. at least 3,000 pg. The total dose for treating paediatric limb spasticity may be 500-56,250 pg, preferably 30,000-56,250 pg. More preferably, the total dose administered for treating paediatric limb spasticity is 3,750-56,250 pg.

The total number of unit doses administered in a given treatment for treating paediatric limb spasticity may be up to 10× the unit dose. In other words, in one embodiment, 10× single unit doses may be administered at 10× injections sites. In another embodiment, 10× single unit doses may be administered at more than 10× injections sites. The total number of unit doses will be divided according to the muscles treated, e.g. 2× unit doses may be administered to the latissimus dorsi, 2× to the subscapularis, 2× to the pectoralis major, and 1× to the flexor carpi ulnaris yielding a total multiple of unit doses administered of 7×. For example, the total number of unit doses administered may be up to 9×, 8×, 7×, 6×, 5×, 4× or 3×. The total number of unit doses administered may be at least 2×, 3×, 4×, 5×, 6×, 7× the unit dose, preferably at least 2×. The total number of unit doses administered may be 2× to 10×, 7× to 10× or 4× to 8×. Preferably, the number of unit doses administered is 10×.

A total dose administered when carrying out the treatment regimen of the present invention for treating paediatric limb spasticity may be up to 4,740 Units. In other words, the total amount of modified BoNT/A administered at a given treatment session for treating paediatric limb spasticity may be up to 4,740 Units. The total dose for treating paediatric limb spasticity may be up to 4,500, 4,000, 3,000, 2,000, 1,000, or 500 Units. Preferably, the total dose for treating paediatric limb spasticity may be up to 4,445 Units of modified BoNT/A. The total dose for treating paediatric limb spasticity may be at least 50, 100, 200, 250, 300, 350, 400, 450, 500, 550, 600, 650, 700, 750, 800, 850, 900, 950, 1000, 1500, 2000, 3000, or 4000 Units. Preferably, the total dose for treating paediatric limb spasticity may be at least 265 Units, more preferably at least 295 Units of modified BoNT/A. The total dose for treating paediatric limb spasticity may be 265-4,740 Units, preferably 295-4,445 Units.

A suitable unit dose for treating paediatric limb spasticity may be 15.5 Units to 353.5 Units of modified BoNT/A and the total dose administered during the treatment is up to 5302.5 Units. An upper limit of the unit dose range for treating paediatric limb spasticity may be 300, 250, 200, 150, 100, 50 or 25 Units of modified BoNT/A, preferably the upper limit is 333 Units. A lower limit of the unit dose range for treating paediatric limb spasticity may be 20, 25, 30, 35, 40, 45, 50, 55, 60, 65, 70, 75, 80, 85, 90, 100, 150, 200, 250, 300, or 325 Units of modified BoNT/A, preferably the lower limit is 21 Units. Preferably, the unit dose of modified BoNT/A for treating paediatric limb spasticity is 21 Units to 333 Units of modified BoNT/A, for example 100 Units to 200 Units. These unit dosages and total dosages may be particularly relevant where the modified BoNT/A comprises a BoNT/A light-chain and translocation domain, and a BoNT/B receptor binding domain (H_{C} domain).

A suitable unit dose for treating paediatric limb spasticity may be 375 pg to 8,500 pg of modified BoNT/A and the total dose administered during the treatment is up to 127,500 pg. An upper limit of the unit dose range for treating paediatric limb spasticity may be 8,000. 7,000, 6,000, 5,000, 4,0000, 3,000, 2,000 or 1,000 pg of modified BoNT/A, preferably the upper limit is 8,000 pg. A lower limit of the unit dose range for treating paediatric limb spasticity may be 400, 450, 500, 550, 600, 650, 700, 750, 800, 850, 900 or 1000 pg of modified BoNT/A, preferably the lower limit is 500 pg. Preferably, the unit dose of modified BoNT/A for treating paediatric limb spasticity is 500 pg to 8,000 pg of modified BoNT/A, e.g. 4,000 pg to 6,000 pg. These unit dosages and total dosages may be particularly relevant where the modified BoNT/A comprises a BoNT/A light-chain and translocation domain, and a BoNT/B receptor binding domain (H_{C} domain).

A total dose administered when carrying out the treatment regimen of the present invention for treating paediatric limb spasticity may be up to 5,302.5 Units. In other words, the total amount of modified BoNT/A administered at a given treatment session for treating paediatric limb spasticity may be up to 5,302.5 Units. The total dose for treating paediatric limb spasticity may be up to 5,000, 4,000, 3,000, 2,000, 1,000 or 500 Units. Preferably, the total dose for treating paediatric limb spasticity may be up to 4,995 Units of modified BoNT/A. The total dose for treating paediatric limb spasticity may be at least 20, 40, 60, 70, 80, 90, 100, 150, 200, 250, 300, 350, 400, 450, 500, 550, 600, 650, 700, 750, 800, 850, 900, 950, 1,000, 1,500, 2,000, 3,000, 4,000, or 5,000 Units. Preferably, the total dose for treating paediatric limb spasticity may be at least 31 Units, more preferably at least 42 Units of modified BoNT/A, e.g. at least 232.5 units. The total dose for treating paediatric limb spasticity may be 42-4995 Units, preferably 232.5-4995 Units. More preferably, the total dose administered is 4725-4995 Units. These total dosages may be particularly relevant where the modified BoNT/A comprises a BoNT/A light-chain and translocation domain, and a BoNT/B receptor binding domain (H_{C} domain).

A total dose administered when carrying out the treatment regimen of the present invention for treating paediatric limb spasticity may be up to 127,500 pg. In other words, the total amount of modified BoNT/A administered at a given treatment session for treating paediatric limb spasticity may be up to 127,500 pg. The total dose for treating paediatric limb spasticity may be up to 127,000, 125,000, 120,000, 110,000 100,000, 80,000, 60,000, 40,000, 30,000, 20,000, or 10,000 pg. Preferably, the total dose for treating paediatric limb spasticity may be up to 120,000 pg of modified BoNT/A. The total dose for treating paediatric limb spasticity may be at least 500, 600, 700, 800, 900, 1,000, 2,000, 3,000, 4,000, 5,000, 7,500, 10,000, 12,500, 15,000, 20,000, 30,000, 40,000, 50,000 or 120,000 pg. Preferably, the total dose for treating paediatric limb spasticity may be at least 750 pg, more preferably at least 1000 pg of modified BoNT/A, e.g. at least 6000 pg. The total dose for treating paediatric limb spasticity may be 1000-120,000 pg, preferably 64,000-120,000 pg. More preferably, the total dose administered for treating paediatric limb spasticity is 7500-120,000 pg. These total dosages may be particularly relevant where the modified BoNT/A comprises a BoNT/A light-chain and translocation domain, and a BoNT/B receptor binding domain (H_{C} domain).

A total dose administered when carrying out the treatment regimen of the present invention for treating paediatric limb spasticity may be up to 3,535 Units. In other words, the total amount of modified BoNT/A administered at a given treatment session for treating paediatric limb spasticity may be up to 3,535 Units. The total dose for treating paediatric limb spasticity may be up to 3,000, 2,000, 1,000, or 500 Units. Preferably, the total dose for treating paediatric limb spasticity may be up to 3,330 Units of modified BoNT/A. The total dose for treating paediatric limb spasticity may be at least 25, 50,100, 200, 250, 300, 350, 400, 450, 500, 550, 600, 650, 700, 750, 800, 850, 900, 950, 1000, 1500, 2000, or 3000 Units. Preferably, the total dose for treating paediatric limb spasticity may be at least 150 Units, more preferably at least 210 Units of modified BoNT/A. The total dose for treating paediatric limb spasticity may be 150-3,535 Units, preferably 210-3330 Units. These unit dosages and total dosages may be particularly relevant where the modified BoNT/A comprises a BoNT/A light-chain and translocation domain, and a BoNT/B receptor binding domain (H_{C} domain).

A modified BoNT/A of the invention preferably has a longer duration of action when compared to unmodified BoNT/A (e.g. Dysport®). Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months.

Administration to the plurality of muscles in accordance with the present invention preferably occurs in the same treatment session.

Treatment may be repeated at an appropriate time period following administration of modified BoNT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

A “subject” as used herein may be a mammal, such as a human or other mammal. Preferably “subject” means a human subject.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

Suitable modified BoNT/A polypeptides (and nucleotide sequences encoding the same, where present) are described in WO 2015/004461 A1 and WO 2017/191315, both of which are incorporated herein by reference in their entirety.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G, as well as those produced by C. baratii and C. butyricum. Said neurotoxins are highly potent and specific and can poison neurons and other cells to which they are delivered. Among the clostridial toxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin) —see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial toxins are non-cytotoxic proteases that cleave SNARE proteins.

The term “H_{C} domain” as used herein means a functionally distinct region of a neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa.

The term “LH_{N} domain” as used herein means a neurotoxin that is devoid of the H_{C} domain and consists of an endopeptidase domain (“L” or “light chain”) and the domain responsible for translocation of the endopeptidase into the cytoplasm (H_{N} domain of the heavy chain).

As discussed above, clostridial toxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Examples of light chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

A preferred modified BoNT/A is one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such modified BoNT/A demonstrates a reduction in, or absence of, side effects compared to the use of known BoNT/A. The increased tissue retention properties of the modified BoNT/A of the invention also provides increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 2, wherein the amino acid residue numbering is determined by alignment with SEQ ID NO: 2. As the presence of a methionine residue at position 1 of SEQ ID NO: 2 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 2 includes a methionine, the position numbering will be as defined above (e.g. ASN 886 will be ASN 886 of SEQ ID NO: 2). Alternatively, where the methionine is absent from SEQ ID NO: 2 the amino acid residue numbering should be modified by −1 (e.g. ASN 886 will be ASN 885 of SEQ ID NO: 2). Similar considerations apply when the methionine at position 1 of the other polypeptide sequences described herein is present/absent, and the skilled person will readily determine the correct amino acid residue numbering using techniques routine in the art.

The amino acid residue(s) indicated for modification are surface exposed amino acid residue(s).

A modified BoNT/A may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277. The modified BoNT/A may be encoded by a nucleic acid sequence having at least 70% sequence identity to a nucleic acid sequence selected from SEQ ID NOs: 3, 5, 7, and 9. For example, a nucleic acid sequence having at least 80%, 90%, 95% or 99.9% sequence identity to a nucleic acid sequence selected from SEQ ID NOs: 3, 5, 7, and 9. Preferably, a modified BoNT/A for use in the invention may be encoded by a nucleic acid comprising (or consisting of) SEQ ID NO: 3, 5, 7 or 9. The modified BoNT/A may comprise a polypeptide sequence having at least 70% sequence identity to a polypeptide sequence selected from SEQ ID NOs: 4, 6, 8, and 10. For example, a polypeptide sequence having at least 80%, 90%, 95% or 99.9% sequence identity to a polypeptide sequence selected from SEQ ID NOs: 4, 6, 8, and 10. Preferably, a modified BoNT/A for use in the invention may comprise (more preferably consist of) a polypeptide sequence selected from SEQ ID NOs: 4, 6, 8, and 10.

The term “one or more amino acid residue(s)” when used in the context of modified BoNT/A preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A may comprise 1-30, 3-20, or 5-10 amino acid modifications. More preferably, the term “one or more amino acid residue(s)” when used in the context of modified BoNT/A means all of the indicated amino acid residue(s).

Preferably, beyond the one or more amino acid modification(s) at the indicated amino acid residue(s), the modified BoNT/A does not contain any further amino acid modifications when compared to SEQ ID NO: 2.

Most preferably, a modified BoNT/A comprises (more preferably consists of) a modification at one or more amino acid residue(s) selected from: ASN 886, ASN 930, SER 955, GLN 991, ASN 1026, ASN 1052, and GLN 1229. The modified BoNT/A may be encoded by a nucleic acid sequence having at least 70% sequence identity to SEQ ID NO: 3. For example, a nucleic acid sequence having at least 80%, 90%, 95% or 99.9% sequence identity to SEQ ID NO: 3. Preferably, a modified BoNT/A for use in the invention may be encoded by a nucleic acid comprising (or consisting of) SEQ ID NO: 3. The modified BoNT/A may comprise a polypeptide sequence having at least 70% sequence identity to SEQ ID NO: 4. For example, a polypeptide sequence having at least 80%, 90%, 95% or 99.9% sequence identity to SEQ ID NO: 4. Preferably, a modified BoNT/A for use in the invention may comprise (more preferably consist of) SEQ ID NO: 4.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A that has an increased positive surface charge and increased isoelectric point when compared to the corresponding unmodified BONT/A.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A of the invention may have a pI value that is at least 0.2, 0.4, 0.5 or 1 μl units higher than that of an unmodified BoNT/A (e.g. SEQ ID NO: 2). Preferably, a modified BoNT/A may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A. In an amino acid substitution, an amino acid residue that forms part of the BoNT/A polypeptide sequence is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Following modification in accordance with the invention, the modified BoNT/A is capable of binding to the target cell receptors that unmodified BoNT/A (e.g. SEQ ID NO: 2) binds.

A modified BoNT/A for use in the invention may comprise a BoNT/A light-chain and translocation domain (a BoNT/A LH_{N} domain), and a BoNT/B H_{C} domain. The BoNT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a BoNT/B polypeptide sequence is provided as SEQ ID NO: 16 (UniProt accession number B1INP5).

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureld=3BTA) and 1 EPW (http://www.rcsb.org/pdb/explore/explore.do?structureld=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology was used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified.
5. The other BoNT serotype sequences were aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID | | | | |
| NO: 16) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8th residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BONT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera is to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BONT/AB chimeras, in particular an increased potency, an increased Safety Ratio and/or a longer duration of action (as well as increased Safety Ratio and/or duration of action when compared to unmodified BoNT/A).

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 16, or a polypeptide sequence having at least 70% sequence identity thereto. The H_{C} domain from BoNT/B may correspond to amino acid residues 860 to 1291 of SEQ ID NO: 16, or a polypeptide sequence having at least 80%, 90% or 95% sequence identity thereto. Preferably, the H_{C} domain from BoNT/B corresponds to amino acid residues 860 to 1291 of SEQ ID NO: 16.

Preferably, the BoNT/AB chimera comprises a BoNT/A1 LH_{N} domain and a BoNT/B1 H_{C} domain. More preferably, the LH_{N} domain corresponds to amino acid residues 1 to 872 of BoNT/A1 (SEQ ID NO: 2) and the H_{C} domain corresponds to amino acid residues 860 to 1291 of BoNT/B1 (SEQ ID NO: 16).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y. Such modifications are present in BoNT/AB chimeras SEQ ID NO: 13 and SEQ ID NO: 14.

The modification may be a modification when compared to unmodified BoNT/B shown as SEQ ID NO: 16, wherein the amino acid residue numbering is determined by alignment with SEQ ID NO: 16. As the presence of a methionine residue at position 1 of SEQ ID NO: 16 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 16 includes a methionine, the position numbering will be as defined above (e.g. E1191 will be E1191 of SEQ ID NO: 16). Alternatively, where the methionine is absent from SEQ ID NO: 16 the amino acid residue numbering should be modified by −1 (e.g. E1191 will be E1190 of SEQ ID NO: 16). Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 11-15. For example, a polypeptide sequence having at least 80%, 90%, 95% or 99.9% sequence identity to a polypeptide sequence selected from SEQ ID NOs: 11-15. Preferably, a modified BoNT/A for use in the invention may comprise (more preferably consist of) a polypeptide sequence selected from SEQ ID NOs: 11-15.

When a modified BoNT/A is a BoNT/AB chimera, it is preferred that the modified BoNT/A comprises a polypeptide sequence having at least 70% sequence identity to SEQ ID NO: 14. For example, a polypeptide sequence having at least 80%, 90%, 95% or 99.9% sequence identity to SEQ ID NO: 14. Preferably, a modified BoNT/A for use in the invention may comprise (more preferably consist of) SEQ ID NO: 14.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

As discussed above, a modified BoNT/A described herein has increased tissue retention properties that also provide increased potency and/or duration of action and can allow for increased dosages without any additional negative effects. One way in which these advantageous properties may be defined is in terms of the Safety Ratio of the modified BoNT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 μl of clostridial toxin, formulated in Gelatin Phosphate Buffer, into the mouse gastrocnemius/soleus complex, followed by assessment of Digital Abduction Score using the method of Aoki (Aoki K R, Toxicon 39:1815-1820; 2001). In the DAS assay, mice are suspended briefly by the tail in order to elicit a characteristic startle response in which the mouse extends its hind limbs and abducts its hind digits. Following clostridial toxin injection, the varying degrees of digit abduction are scored on a five-point scale (0=normal to 4=maximal reduction in digit abduction and leg extension).

The Safety Ratio of a modified BoNT/A of the invention (or unmodified BoNT/A for comparison) may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A modified BoNT/A of the present invention has a Safety Ratio that is higher than the Safety Ratio of an equivalent unmodified (native) BoNT/A.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for-10% bodyweight change (pg/mouse) divided by DAS ED₅₀ (pg/mouse) [ED₅₀=dose required to produce a DAS score of 2].

In one embodiment, a modified BoNT/A of the present invention has a Safety Ratio of at least 10. In one embodiment, a modified BoNT/A of the present invention has a Safety Ratio of at least 15.

Preferably, where a modified BoNT/A is one comprising one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277 as described herein, said modified BoNT/A has a Safety Ratio of at least 20, more preferably at least 22 (e.g. 23-25).

Preferably, where a modified BoNT/A is one that comprises a BoNT/A light-chain and translocation domain, and a BoNT/B H_{C} domain, the modified BoNT/A has a Safety Ratio of at least 10, more preferably at least 12 (e.g. 14-15).

In use, the modified BoNT/A of the invention is in a di-chain form.

The modified BoNT/A is preferably in a non-complexed form (i.e. free from complexing proteins that are present in naturally occurring BoNT/A). Examples of such complexing proteins include a neurotoxin-associated proteins (NAP) and a nontoxic-nonhemagglutinin component (NTNH). It is preferred that the modified BoNT/A is a recombinant modified BoNT/A.

The modified BoNT/A may be produced by a method of producing a single-chain modified BoNT/A having a light chain and a heavy chain, the method comprising expressing a nucleic acid (said nucleic acid being as described above) in a suitable host cell, lysing the host cell to provide a host cell homogenate containing the single-chain modified BoNT/A, and isolating the single-chain modified BoNT/A. The modified BoNT/A may then be activated by a method comprising providing a single-chain modified BoNT/A protein obtainable by the method of producing a single-chain modified BoNT/A as described above, contacting the modified BoNT/A with a protease that cleaves the polypeptide at a recognition site (cleavage site) located between the light chain and heavy chain, thereby converting the polypeptide into a di-chain modified BoNT/A wherein the light chain and heavy chain are joined together by a disulphide bond.

The modified BoNT/A of the invention may be formulated in any suitable manner for administration to a subject, for example as part of a pharmaceutical composition. Thus, in one aspect, the invention provides a pharmaceutical composition comprising a modified BoNT/A of the invention and a pharmaceutically acceptable carrier, excipient, adjuvant, and/or salt.

In one aspect, the invention provides a unit dosage form of modified botulinum neurotoxin A (BoNT/A), the unit dosage form comprising:
a. 53 Units to 948 Units of modified BoNT/A, wherein 1 Unit is an amount of the modified BoNT/A that corresponds to the calculated median lethal dose (LD₅₀) in mice; or
b. 450 pg to 8,000 pg of modified BoNT/A; and
c. optionally a pharmaceutically acceptable carrier, excipient, adjuvant, and/or salt, wherein the modified BoNT/A comprises a modification at one or more amino acid residue(s) selected from: 886, 905, 915, 918, 920, 930, 954, 955, 991, 992, 995, 1006, 1025, 1026, 1032, 1043, 1046, 1052, 1058, 1064, 1080, 1081, 1083, 1086, 1188, 1213, 1215, 1216, 1229, 1242, 1243, 1274, and 1277, when compared to unmodified BoNT/A shown as SEQ ID NO: 2, wherein the amino acid residue numbering is determined by alignment with SEQ ID NO: 2, and wherein the modification is selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

In one aspect, the invention provides a unit dosage form of modified botulinum neurotoxin A (BoNT/A), the unit dosage form comprising:
a. 53 Units to 948 Units of modified BoNT/A, wherein 1 Unit is an amount of the modified BoNT/A that corresponds to the calculated median lethal dose (LD₅₀) in mice; or
b. 450 pg to 8,000 pg of modified BoNT/A; and
c. optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain).

A unit dosage form may comprise 53 Units to 948 Units of modified BoNT/A. An upper limit said range may be 925, 900, 850, 800, 750, 700, 650, 600, 550, 500, 450, 400, 350, 300, 250, 200, 150 or 100 Units of modified BoNT/A, preferably the upper limit is 889 Units. A lower limit of said range may be 55, 60, 65, 70, 75, 80, 85, 90, 100, 150, 200, 250, 300, 350, 400, 450, 500, 550, 600, 650, 700, 750, 800, 850 or 900 Units of modified BoNT/A, preferably the lower limit is 59 Units. Preferably, a unit dosage form comprises 59 Units to 889 Units of modified BoNT/A, for example 200 Units to 600 Units.

A unit dosage form may comprise 450 pg to 8,000 pg of modified BoNT/A. An upper limit of said range may be 7,750, 7,500, 7,000, 6,000, 5,000, 4,000, 3,000, 2,000 or 1,000, pg of modified BoNT/A, preferably the upper limit is 7,500 pg. A lower limit of said range may be 475, 500, 600, 700, 800, 900, 1,000, 1,500, 2,000, 3,000, 4,000, 5,000, 6,000 or 7,000 pg of modified BoNT/A, preferably the lower limit is 500 pg. Preferably, a unit dosage form comprises 500 pg to 7,500 pg of modified BoNT/A, e.g. 4,000 pg to 6,000 pg.

In another aspect, the invention provides a unit dosage form of modified botulinum neurotoxin A (BoNT/A), the unit dosage form comprising:
a. 31 Units to 707 Units of modified BoNT/A, wherein 1 Unit is an amount of the modified BoNT/A that corresponds to the calculated median lethal dose (LD₅₀) in mice; or
b. 750 pg to 17,000 pg of modified BoNT/A; and
c. optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain)

A unit dosage form may comprise 31 Units to 707 Units of modified BoNT/A. An upper limit said range may be 700, 650, 600, 550, 500, 450, 400, 350, 300, 250, 200, 150 or 100 Units of modified BoNT/A, preferably the upper limit is 666 Units. A lower limit of said range may be 40, 45, 50, 60, 65, 70, 75, 80, 85, 90, 100, 150, 200, 250, 300, 350, 400, 450, 500, 550, 600, 650, or 700 Units of modified BoNT/A, preferably the lower limit is 42 Units. Preferably, a unit dosage form comprises 42 Units to 666 Units of modified BoNT/A, for example 200 Units to 400 Units.

A unit dosage form may comprise 750 pg to 17,000 pg of modified BoNT/A. An upper limit of said range may be 16,500, 15,500, 14,500, 13,500, 12,500, 11,500, 10,500, 9,500, 8,500, 7,500, 6,500, 5,500, 4,500, 3,500, 2,500, 1,500 or 500 pg of modified BoNT/A, preferably the upper limit is 16,000 pg. A lower limit of said range may be 750, 850, 950, 1000, 1500, 2000, 2,500, 3,000, 3,500, 4,000, 4,500 or 5,000 pg of modified BoNT/A, preferably the lower limit is 1000 pg.

Preferably, a unit dosage form comprises 1000 pg to 16,000 pg of modified BoNT/A, e.g. 4,000 pg to 6,000 pg.

As discussed earlier, the unit dose for paediatric applications may be 50% or less than that indicated above. In some embodiments, the unit dose is typically 70% or less (e.g. 67% or less) than that used when treating adult limb spasticity.

In one aspect, the invention provides a unit dosage form of modified botulinum neurotoxin A (BoNT/A), the unit dosage form comprising:
a. 26.5 Units to 474 Units of modified BoNT/A, wherein 1 Unit is an amount of the modified BoNT/A that corresponds to the calculated median lethal dose (LD₅₀) in mice; or
b. 225 pg to 4,000 pg of modified BoNT/A; and
c. optionally a pharmaceutically acceptable carrier, excipient, adjuvant, and/or salt, wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another aspect, the invention provides a unit dosage form of modified botulinum neurotoxin A (BoNT/A), the unit dosage form comprising:
a. 26.5 Units to 474 Units of modified BoNT/A, wherein 1 Unit is an amount of the modified BoNT/A that corresponds to the calculated median lethal dose (LD₅₀) in mice; or
b. 225 pg to 4,000 pg of modified BoNT/A; and
c. optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain).

A unit dosage form may comprise 26.5 Units to 474 Units of modified BoNT/A. An upper limit said range may be 425, 400, 350, 300, 250, 200, 150, 100 or 50 Units of modified BoNT/A, preferably the upper limit is 444.5 Units. A lower limit of said range may be 30, 35, 40, 45, 50, 55, 60, 65, 70, 75, 80, 85, 90, 95, 100, 150, 200, 250, 300, 350, 400, 450, 500, 550 or 600 Units of modified BoNT/A, preferably the lower limit is 29.5 Units. Preferably, a unit dosage form comprises 29.5 Units to 444.5 Units of modified BoNT/A, for example 100 Units to 300 Units.

A unit dosage form may comprise 225 pg to 4,000 pg of modified BoNT/A. An upper limit of said range may be 3,750, 3,500, 3,000, 2,000, 1,000, 4,000, or 500 pg of modified BoNT/A, preferably the upper limit is 3,750 pg. A lower limit of said range may be 225, 250, 300, 400, 500, 600, 800, 1,000, 1,500, 2,000, 2,500, 3000 or 3500 pg of modified BoNT/A, preferably the lower limit is 250 pg. Preferably, a unit dosage form comprises 250 pg to 3,750 pg of modified BoNT/A, e.g. 2,000 pg to 3,000 pg.

In another aspect, the invention provides a unit dosage form of modified botulinum neurotoxin A (BoNT/A), the unit dosage form comprising:
a. 15.5 Units to 353.5 Units of modified BoNT/A, wherein 1 Unit is an amount of the modified BoNT/A that corresponds to the calculated median lethal dose (LD₅₀) in mice; or
b. 375 pg to 8,500 pg of modified BoNT/A; and
c. optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain)

A unit dosage form may comprise 15.5 Units to 353.5 Units of modified BoNT/A. An upper limit said range may be 350, 300, 250, 200, 150, 100, or 50 Units of modified BoNT/A, preferably the upper limit is 333 Units. A lower limit of said range may be 20, 25, 30, 35, 40, 45, 50, 65, 70, 75, 80, 85, 90, 95, or 100Units of modified BoNT/A, preferably the lower limit is 21 Units. Preferably, a unit dosage form comprises 21 Units to 33 Units of modified BoNT/A, for example 200 Units to 400 Units.

A unit dosage form may comprise 375 pg to 8,500 pg of modified BoNT/A. An upper limit of said range may be 7,500, 7,000, 6,500, 6,000, 5,500, 5,000, 4,500, 4,000, 3,500, 3,000, 2,500, 2,000, 1,500, 1,000, or 500 pg of modified BoNT/A, preferably the upper limit is 8,000 pg. A lower limit of said range may be 450, 550, 650, 750, 850, 950, 1,000, 1,500, 2,000, 2,500 or 3,000 pg of modified BoNT/A, preferably the lower limit is 500 pg. Preferably, a unit dosage form comprises 500 pg to 8,000 pg of modified BoNT/A, e.g. 2000 pg to 4,000 pg.

In another aspect, the invention provides a kit comprising:
a. the unit dosage form according to the present invention; and
b. instructions for use of the same in treating limb spasticity; and
c. optionally a diluent.

Embodiments related to the various therapeutic uses of the invention can be applied to the methods, compositions (e.g. unit dosage forms), and kits of the invention and vice versa.

### SEQUENCE HOMOLOGY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}\begin{matrix}length of the longer sequence plus the \\ number of gaps introduced into the longer\end{matrix} \\ sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylaline |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a botulinum neurotoxin A” includes a plurality of such candidate agents and reference to “the botulinum neurotoxin A” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the five most common upper limb spasticity clinical patterns involving upper arm joints.

FIG. 2 shows the FDA approved dosages of Dysport® for treating spasticity in adults.

FIG. 3 shows the isoelectric focusing (IEF) gel of cationic constructs.

FIGS. 4A-4D show the percentage SNAP-25 cleavage in rat embryonic spinal cord neurons (eSCN) for Cat5v2(K1064H/N954K) (FIG. 4A), Cat5v2(K1064H/N886K) (FIG. 4B) and Cat5v2(K1064H/N1025K) (FIG. 4C), and summary of pEC50 relative to nBoNT/A1. (FIGS. 4A, 4B, and 4C) Rat embryonic spinal cord neurons were cultured for three weeks and treated with Cat5v4 for 24 h, before Western blotting with SNAP-25 specific antibody. Data is mean±SEM from three independent experiments in triplicate. (FIG. 4D) Relative potency of Cat5v2(K1064H/N886K), Cat5v2(K1064H/N954K) and Cat5v2(K1064H/N1025K) to nBoNT/A1 (List Biological Laboratories) in the rat eSCN SNAP-25 cleavage potency assay. Each point corresponds to an individual batch and is a mean of 3 independent pEC50 determinations based on an 8-point concentration response curve (CRC). Each concentration in the CRC was assessed in triplicate. Potency comparisons are made to a mean of List batches, pooled data n=24. Data are mean±SEM of n=3 batches per Cat5v4.

FIG. 5 shows the potency (t₅₀) of nBoNT/A1 and Cat5v4 in the mouse phrenic nerve hemi-diaphragm assay (mPNHD). Mouse phrenic nerve hemi-diaphragm tissue was incubated with Cat5v4 or native BoNT/A1 as indicated. Diaphragm contractile force was recorded until the contraction was no longer detectable or after 140 minutes. Each point corresponds to independent determinations. The t₅₀ value is the time required to inhibit the contractile force of the mouse hemi-diaphragm by 50%.

FIG. 6 shows SDS-PAGE of purified recombinant BoNT/AB chimera 1, 2 and 3A (SEQ ID NO: 11, 12 and 13 respectively). Lanes are labelled “Marker” (molecular weight marker), “−DTT” (oxidised BoNT/AB chimera sample), and “+DTT” (reduced BoNT/AB chimera sample).

FIG. 7 shows cleavage of SNAP-25 in rat spinal cord neurones by recombinant BoNT/AB chimera 1, 2 and 3A (SEQ ID NO: 11, 12 and 13 respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 8 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIG. 9 shows SDS-PAGE of purified recombinant BoNT/AB chimera 3B and 3C (SEQ ID NO: 14 and 15 respectively). Lanes are labelled “Marker” (molecular weight marker), “−DTT” (oxidised BoNT/AB chimera sample), and “+DTT” (reduced BoNT/AB chimera sample).

FIG. 10 shows cleavage of SNAP-25 by unmodified BoNT/A and BoNT/AB chimera 3B and 3C (SEQ ID NO: 2, 14 and 15 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 11 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

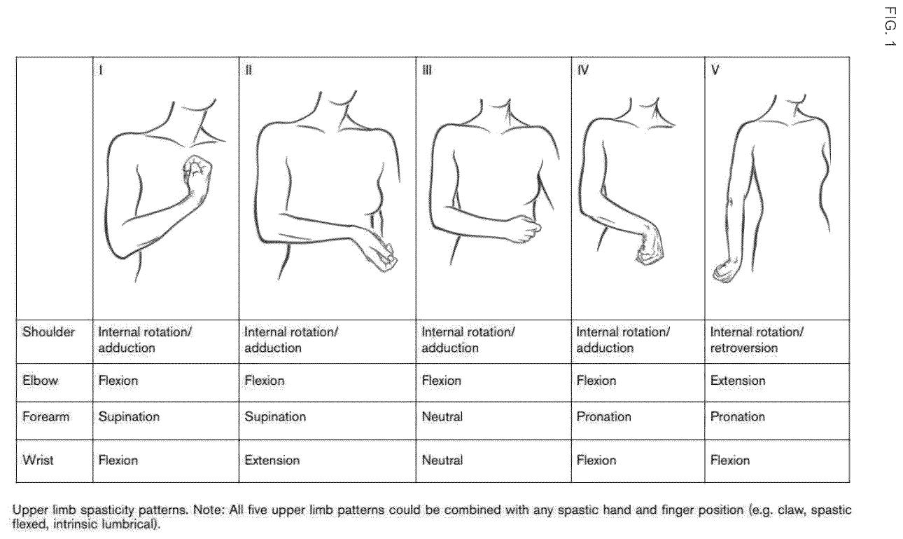

### SEQUENCE LISTING

Where an initial Met amino acid residue or a corresponding initial codon is indicated in any of the following SEQ ID NOs, said residue/codon is optional.

(Nucleotide Sequence of Unmodified BoNT/A)
SEQ ID NO: 1
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTGGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGA
GATTTACACCGAGGACAACTTTGTGAAATTCTTCAAAGTGTTGAATCGTAAAACCTATCTGAATT
TTGACAAAGCGGTTTTCAAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAATTGCTG
ATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCAATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAATCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAATCATCTGA
TTGATCTGAGCCGTTATGCAAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAACGCCAT
TGTCTACAACTCCATGTACGAGAATTTCTCCACCAGCTTCTGGATTCGCATCCCGAAATACTTCA
ACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGT
CGTGTTCAAGTACTCTCAAATGATCAACATTTCCGATTACATTAATCGTTGGATCTTCGTGACCA
TTACGAATAACCGTCTGAATAACAGCAAGATTTACATCAATGGTCGCTTGATCGATCAGAAACCG
ATTAGCAACCTGGGTAATATCCACGCAAGCAACAACATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGAATAAAGAGTACCG
TCTGGCGACCAACGCTTCGCAGGCGGGTGTTGAGAAAATTCTGAGCGCGTTGGAGATCCCTGATG
TCGGTAATCTGAGCCAAGTCGTGGTTATGAAGAGCAAGAACGACCAGGGTATCACTAACAAGTGC
AAGATGAACCTGCAAGACAACAATGGTAACGACATCGGCTTTATTGGTTTCCACCAGTTCAACAA
TATTGCTAAACTGGTAGCGAGCAATTGGTACAATCGTCAGATTGAGCGCAGCAGCCGTACTTTGG
GCTGTAGCTGGGAGTTTATCCCGGTCGATGATGGTTGGGGCGAACGTCCGCTG
(Polypeptide Sequence of Unmodified BoNT/A)
SEQ ID NO: 2
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESNHLIDLSRYASKINIGSKVNFDPID
KNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKP
ISNLGNIHASNNIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDQGITNKC
KMNLQDNNGNDIGFIGFHQFNNIAKLVASNWYNRQIERSSRTLGCSWEFIPVDDGWGERPL
(Nucleotide Sequence of Modified BoNT/A “Cat-A”)
SEQ ID NO: 3
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTGGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGA
GATTTACACCGAGGACAACTTTGTGAAATTCTTCAAAGTGTTGAATCGTAAAACCTATCTGAATT
TTGACAAAGCGGTTTTCAAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAATTGCTG
ATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCAATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAATCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAAGCATCTGA
TTGATCTGAGCCGTTATGCTAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAAGGCCAT
TGTCTACAACTCCATGTACGAGAATTTCTCCACCAGCTTCTGGATTCGCATCCCGAAATACTTCA
ACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGT
CGTGTTCAAGTACTCTCAAATGATCAACATTTCCGATTACATTAATCGTTGGATCTTCGTGACCA
TTACGAATAACCGTCTGAATAAGAGCAAGATTTACATCAATGGTCGCTTGATCGATCAGAAACCG
ATTAGCAACCTGGGTAATATCCACGCAAGCAACAAGATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGAATAAAGAGTACCG
TCTGGCGACCAACGCTTCGCAGGCGGGTGTTGAGAAAATTCTGAGCGCGTTGGAGATCCCTGATG
TCGGTAATCTGAGCCAAGTCGTGGTTATGAAGAGCAAGAACGACAAGGGTATCACTAACAAGTGC
AAGATGAACCTGCAAGACAACAATGGTAACGACATCGGCTTTATTGGTTTCCACCAGTTCAACAA
TATTGCTAAACTGGTAGCGAGCAATTGGTACAATCGTCAGATTGAGCGCAGCAGCCGTACTTTGG
GCTGTAGCTGGGAGTTTATCCCGGTCGATGATGGTTGGGGCGAACGTCCGCTG
(Polypeptide Sequence of Modified BoNT/A “Cat-A”)
SEQ ID NO: 4
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESKHLIDLSRYASKINIGSKVNFDPID
KNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKP
ISNLGNIHASNKIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDKGITNKC
KMNLQDNNGNDIGFIGFHQFNNIAKLVASNWYNRQIERSSRTLGCSWEFIPVDDGWGERPL
(Nucleotide Sequence of Modified BoNT/A “Cat-B”)
SEQ ID NO: 5
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTGGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACaAGATGCTGACCGA
GATTTACACCGAGGACAACTTTGTGAAATTCTTCAAAGTGTTGAATCGTAAAACCTATCTGAATT
TTGACAAAGCGGTTTTCAAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAATTGCTG
ATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCAATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAaTCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAATCATCTGA
TTGATCTGAGCCGTTATGCTAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAAGGCCAT
TGTCTACAACTCCATGTACGAGAATTTCTCCACCAGCTTCTGGATTCGCATCCCGAAATACTTCA
AGAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGT
CGTGTTCAAGTACTCTCAAATGATCAACATTTCCGATTACATTAATCGTTGGATCTTCGTGACCA
TTACGAATAACCGTCTGAATAAGAGCAAGATTTACATCAATGGTCGCTTGATCGATCAGAAACCG
ATTAGCAACCTGGGTAATATCCACGCAAGCAACAAGATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGAATAAAGAGTACCG
TCTGGCGACCAACGCTTCGCAGGCGGGTGTTGAGAAAATTCTGAGCGCGTTGGAGATCCCTGATG
TCGGTAATCTGAGCCAAGTCGTGGTTATGAAGAGCAAGAACGACAAGGGTATCACTAACAAGTGC
AAGATGAACCTGCAAGACAACAATGGTAACGACATCGGCTTTATTGGTTTCCACCAGTTCAACAA
TATTGCTAAACTGGTAGCGAGCAATTGGTACAATCGTCAGATTGAGCGCAGCAGCCGTACTTTGG
GCTGTAGCTGGGAGTTTATCCCGGTCGATGATGGTTGGGGCGAACGTCCGCTG
(Polypeptide Sequence of Modified BoNT/A “Cat-B”)
SEQ ID NO: 6
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESNHLIDLSRYASKINIGSKVNFDPID
KNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKP
ISNLGNIHASNKIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDKGITNKC
KMNLQDNNGNDIGFIGFHQFNNIAKLVASNWYNRQIERSSRTLGCSWEFIPVDDGWGERPL
(Nucleotide Sequence of Modified BoNT/A “Cat-C”)
SEQ ID NO: 7
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTGGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGA
GATTTACACCGAGGACAACTTTGTGAAATTCTTCAAAGTGTTGAATCGTAAAACCTATCTGAATT
TTGACAAAGCGGTTTTCAAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAATTGCTG
ATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCAATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAATCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAATCATCTGA
TTGATCTGAGCCGTTATGCTAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAAGGCCAT
TGTCTACAACTCCATGTACGAGAATTTCTCCACCAGCTTCTGGATTCGCATCCCGAAATACTTCA
ACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGT
CGTGTTCAAGTACTCTCAAATGATCAACATTTCCGATTACATTAATCGTTGGATCTTCGTGACCA
TTACGAATAACCGTCTGAAGAAGAGCAAGATTTACATCAATGGTCGCTTGATCGATCAGAAACCG
ATTAGCAACCTGGGTAATATCCACGCAAGCAACAAGATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGAATAAAGAGTACCG
TCTGGCGACCAACGCTTCGCAGGCGGGTGTTGAGAAAATTCTGAGCGCGTTGGAGATCCCTGATG
TCGGTAATCTGAGCCAAGTCGTGGTTATGAAGAGCAAGAACGACAAGGGTATCACTAACAAGTGC
AAGATGAACCTGCAAGACAACAATGGTAACGACATCGGCTTTATTGGTTTCCACCAGTTCAACAA
TATTGCTAAACTGGTAGCGAGCAATTGGTACAATCGTCAGATTGAGCGCAGCAGCCGTACTTTGG
GCTGTAGCTGGGAGTTTATCCCGGTCGATGATGGTTGGGGCGAACGTCCGCTG
(Polypeptide Sequence of Modified BoNT/A “Cat-C”)
SEQ ID NO: 8
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESNHLIDLSRYASKINIGSKVNFDPID
KNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKP
ISNLGNIHASNKIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDKGITNKC
KMNLQDNNGNDIGFIGFHQFNNIAKLVASNWYNRQIERSSRTLGCSWEFIPVDDGWGERPL
(Nucleotide Sequence of Modified BoNT/A “Cat-D”)
SEQ ID NO: 9
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTgGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGA
GATTTACACCGAGGACAACTTTGTGAAATTCTTCAAaGTGTTGAATCGTAAAACCTATCTGAATT
TTGACAAAGCGGTTTTCaAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAaTTGCTG
ATaTTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCaATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAATCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAATCATCTGA
TIGATCTGAGCCGTTATGCAAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAACGCCAT
TGTCTACAACTCCATGTACGAGAATTTCTCCACCAGCTTCTGGATTCGCATCCCGAAATACTTCA
ACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGT
CGTGTTCAAGTACTCTCAAATGATCAACATTTCCGATTACATTAATCGTTGGATCTTCGTGACCA
TTACGAATAACCGTCTGAATAACAGCAAGATTTACATCAATGGTCGCTTGATCGATCAGAAACCG
ATTAGCAACCTGGGTAATATCCACGCAAGCAACAACATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGCGTAAAGAGTACCG
TCTGGCGACCAACGCTTCGCAGGCGGGTGTTGAGAAAATTCTGAGCGCGTTGGAGATCCCTCGTG
TCCGTCGTCTGAGCCAAGTCGTGGTTATGAAGAGCAAGAACGACCAGGGTATCACTAACAAGTGC
AAGATGAACCTGCAAGACCGTCGTGGTAACGACATCGGCTTTATTGGTTTCCACCAGTTCAACAA
TATTGCTAAACTGGTAGCGAGCAATTGGTACAATCGTCAGATTGAGCGCCGTAGCCGTCGTTTGG
GCTGTAGCTGGGAGTTTATCCCGGTCGATGATGGTTGGGGCGAACGTCCGCTG
(Polypeptide Sequence of Modified BoNT/A “Cat-D”)
SEQ ID NO: 10
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESNHLIDLSRYASKINIGSKVNFDPID
KNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKP
ISNLGNIHASNNIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKRKEYRLATNASQAGVEKILSALEIPRVRRLSQVVVMKSKNDQGITNKC
KMNLQDRRGNDIGFIGFHQFNNIAKLVASNWYNRQIERRSRRLGCSWEFIPVDDGWGERPL
(Polypeptide Sequence of Modified BoNT/A “Chimera 1”)
SEQ ID NO: 11
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKSEILNNIILNLRYKDNNLIDLSGYGAKVEVYDGVELNDK
NQFKLTSSANSKIRVTQNQNIIFNSVFLDFSVSFWIRIPKYKNDGIQNYIHNEYTIINCMKNNSG
WKISIRGNRIIWTLIDINGKTKSVFFEYNIREDISEYINRWFFVTITNNLNNAKIYINGKLESNT
DIKDIREVIANGEIIFKLDGDIDRTQFIWMKYFSIFNTELSQSNIEERYKIQSYSEYLKDFWGNP
LMYNKEYYMFNAGNKNSYIKLKKDSPVGEILTRSKYNQNSKYINYRDLYIGEKFIIRRKSNSQSI
NDDIVRKEDYIYLDFFNLNQEWRVYTYKYFKKEEMKLFLAPIYDSDEFYNTIQIKEYDEQPTYSC
QLLFKKDEESTDEIGLIGIHRFYESGIVFEEYKDYFCISKWYLKEVKRKPYNLKLGCNWQFIPKD
EGWTEHHHHHHH
(Polypeptide Sequence of Modified BoNT/A “Chimera 2”)
SEQ ID NO: 12
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIIELGGGGSELSEILNNIILNLRYKDNNLIDLSGYGAK
VEVYDGVELNDKNQFKLTSSANSKIRVTQNQNIIFNSVFLDFSVSFWIRIPKYKNDGIQNYIHNE
YTIINCMKNNSGWKISIRGNRIIWTLIDINGKTKSVFFEYNIREDISEYINRWFFVTITNNLNNA
KIYINGKLESNTDIKDIREVIANGEIIFKLDGDIDRTQFIWMKYFSIFNTELSQSNIEERYKIQS
YSEYLKDFWGNPLMYNKEYYMFNAGNKNSYIKLKKDSPVGEILTRSKYNQNSKYINYRDLYIGEK
FIIRRKSNSQSINDDIVRKEDYIYLDFFNLNQEWRVYTYKYFKKEEMKLFLAPIYDSDEFYNTIQ
IKEYDEQPTYSCQLLFKKDEESTDEIGLIGIHRFYESGIVFEEYKDYFCISKWYLKEVKRKPYNL
KLGCNWQFIPKDEGWTEHHHHHHHHHH
(Polypeptide Sequence of Modified BoNT/A “Chimera 3A”)
SEQ ID NO: 13
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMFNAGNKNSYIKLKKDSPVGEILTRSKYNQNSKYINYRDLYIGEKFIIRRKSNSQSIN
DDIVRKEDYIYLDFFNLNQEWRVYTYKYFKKEEMKLFLAPIYDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTEHHHHHHHHHH
(Polypeptide Sequence of Modified BoNT/A “Chimera 3B”)
SEQ ID NO: 14
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMFNAGNKNSYIKLKKDSPVGEILTRSKYNQNSKYINYRDLYIGEKFIIRRKSNSQSIN
DDIVRKEDYIYLDFFNLNQEWRVYTYKYFKKEEMKLFLAPIYDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTE
(Polypeptide Sequence of Modified BoNT/A “Chimera 3C”)
SEQ ID NO: 15
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMFNAGNKNSYIKLKKDSPVGEILTRSKYNQNSKYINYRDLYIGEKFIIRRKSNSQSIN
DDIVRKEDYIYLDFFNLNQEWRVYTYKYFKKEEEKLFLAPISDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTE
(Polypeptide Sequence of BoNT/B)
SEQ ID NO: 16
MPVTINNFNYNDPIDNNNIIMMEPPFARGTGRYYKAFKITDRIWIIPERYTFGYKPEDFNKSSGI
FNRDVCEYYDPDYLNTNDKKNIFLQTMIKLFNRIKSKPLGEKLLEMIINGIPYLGDRRVPLEEFN
TNIASVTVNKLISNPGEVERKKGIFANLIIFGPGPVLNENETIDIGIQNHFASREGFGGIMQMKF
CPEYVSVFNNVQENKGASIFNRRGYFSDPALILMHELIHVLHGLYGIKVDDLPIVPNEKKFFMQS
TDAIQAEELYTFGGQDPSIITPSTDKSIYDKVLQNFRGIVDRLNKVLVCISDPNININIYKNKFK
DKYKFVEDSEGKYSIDVESFDKLYKSLMFGFTETNIAENYKIKTRASYFSDSLPPVKIKNLLDNE
IYTIEEGFNISDKDMEKEYRGQNKAINKQAYEEISKEHLAVYKIQMCKSVKAPGICIDVDNEDLF
FIADKNSFSDDLSKNERIEYNTQSNYIENDFPINELILDTDLISKIELPSENTESLTDFNVDVPV
YEKQPAIKKIFTDENTIFQYLYSQTFPLDIRDISLTSSFDDALLFSNKVYSFFSMDYIKTANKVV
EAGLFAGWVKQIVNDFVIEANKSNTMDKIADISLIVPYIGLALNVGNETAKGNFENAFEIAGASI
LLEFIPELLIPVVGAFLLESYIDNKNKIIKTIDNALTKRNEKWSDMYGLIVAQWLSTVNTQFYTI
KEGMYKALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPFDLSIYTN
DTILIEMFNKYNSEILNNIILNLRYKDNNLIDLSGYGAKVEVYDGVELNDKNQFKLTSSANSKIR
VTQNQNIIFNSVFLDFSVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEI
IFKLDGDIDRTQFIWMKYFSIFNTELSQSNIEERYKIQSYSEYLKDFWGNPLMYNKEYYMFNAGN
KNSYIKLKKDSPVGEILTRSKYNQNSKYINYRDLYIGEKFIIRRKSNSQSINDDIVRKEDYIYLD
FFNLNQEWRVYTYKYFKKEEEKLFLAPISDSDEFYNTIQIKEYDEQPTYSCQLLFKKDEESTDEI
GLIGIHRFYESGIVFEEYKDYFCISKWYLKEVKRKPYNLKLGCNWQFIPKDEGWTE

### EXAMPLES

### Example 1

### Cloning, Expression and Purification

The nucleotide sequence SEQ ID NO: 1, which encodes wild-type BoNT/A (SEQ ID NO: 2) was mutated to introduce the following substitutions to form the four constructs shown in Table 1 below:

TABLE 1
Constructs.

| | | Nucleotide | Polypeptide |
|---|---|---|---|
| Construct | Mutations | Sequence | Sequence |
| Cat-A | N930K, S955K, | 3 | 4 |
| | Q991K, N1026K, | | |
| | N1052K, Q1229K, | | |
| | N886K | | |
| Cat-B | N930K, S955K, | 5 | 6 |
| | Q991K, N1026K, | | |
| | N1052K, Q1229K, | | |
| | N954K | | |
| Cat-C | N930K, S955K, | 7 | 8 |
| | Q991K, N1026K, | | |
| | N1052K, Q1229K, | | |
| | N1025K | | |
| Cat-D* | N1188R, D1213R, | 9 | 10 |
| | G1215R, N1216R, | | |
| | N1242R, N1243R, | | |
| | S1274R, T1277R | | |

*Cat-D had a calculated pI of 7.45, and a molecular weight of 149,859.

DNA constructs encoding the modified BoNT/A molecules above were synthesised, cloned into the pJ401 expression vector and then transformed into BL21 (DE3) E. coli. This allowed for soluble over-expression of the recombinant Cat-A, Cat-B, Cat-C, and Cat-D proteins in BL21 (DE3) E. coli.

The recombinant modified BoNTs were purified using classical chromatography techniques from the E. coli lysates. An initial purification step using a cation-exchange resin was employed, followed by an intermediate purification step using a hydrophobic interaction resin. The recombinant modified BONT single-chain was then cleaved by proteolysis, resulting in the activated di-chain modified BoNT. A final purification step was then employed to remove remaining contaminants. Suitable techniques are taught in WO2015/166242, WO2017055274A1, EP2524963B1, EP2677029B1, and U.S. Pat. No. 10,087,432B2.

### Example 2

### Characterization of Purified Modified BoNT/A

The modified BoNTs described in Example 1 above were characterised experimentally as follows.

Measurement of the pI showed that the modified BoNTs had an isoelectric point greater than that of unmodified (native) BoNT/A1—see FIG. 3 and Table 2 below.

TABLE 2
Modified BoNT/A pI values.

| | pI | pI |
|---|---|---|
| BoNT/A1 molecule | (calculated) | (observed) |
| Modified, “Cat-A” | 6.9 | ~8.0 |
| [Cat5v2(K1064H/N886K] | | |
| (SEQ ID NO: 4) | | |
| Modified, “Cat-B” | 6.9 | ~8.0 |
| [Cat5v2(K1064/N954K)] | | |
| (SEQ ID NO: 6) | | |
| Modified, “Cat-C” | 6.9 | 7.8-8.0 |
| [Cat5v2(K1064H/N1025K)] | | |
| (SEQ ID NO: 8) | | |
| Native BoNT/A1 | 6.05 | ~7.4 |
| [rBoNT/A1] | | |
| (SEQ ID NO: 2) | | |

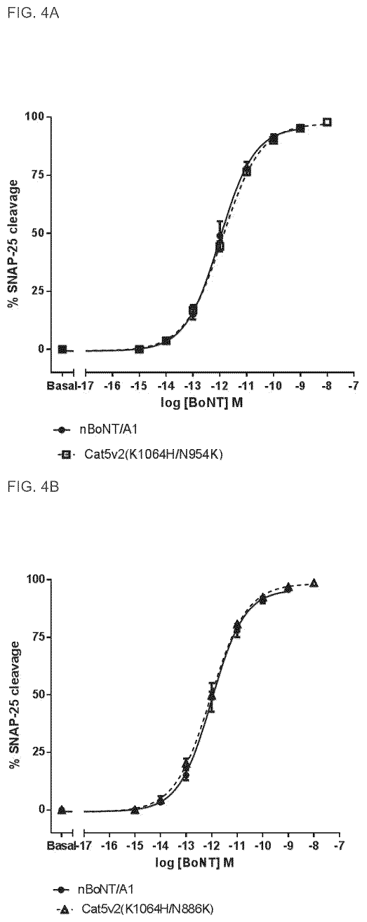

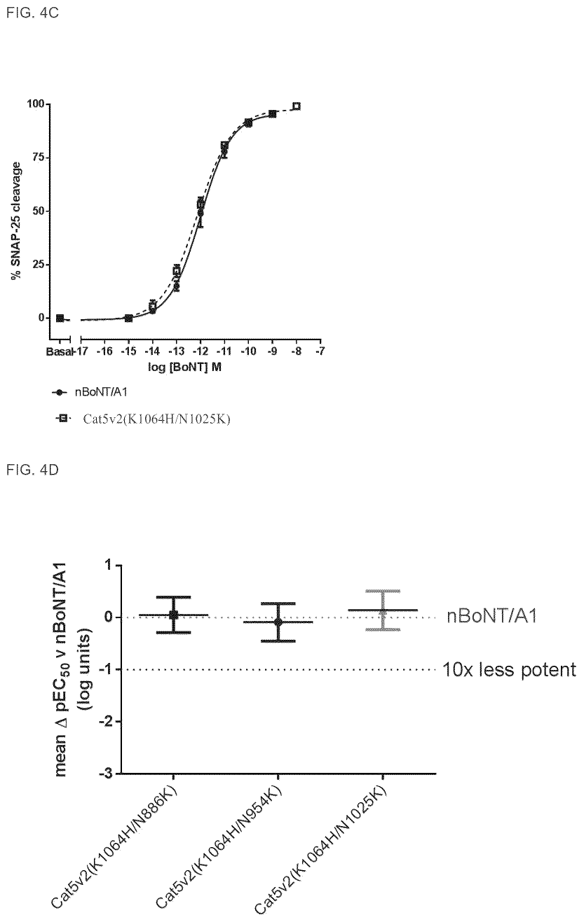

The ability of the modified BoNTs to enter neurons and cleave SNAP-25 (the target of BoNT/A1) was assessed using rat embryonic spinal cord neurons (eSCN). FIGS. 4A-4D show that the modified BoNTs retained the same ability to enter the neuron and cleave SNAP-25 as native BoNT/A1.

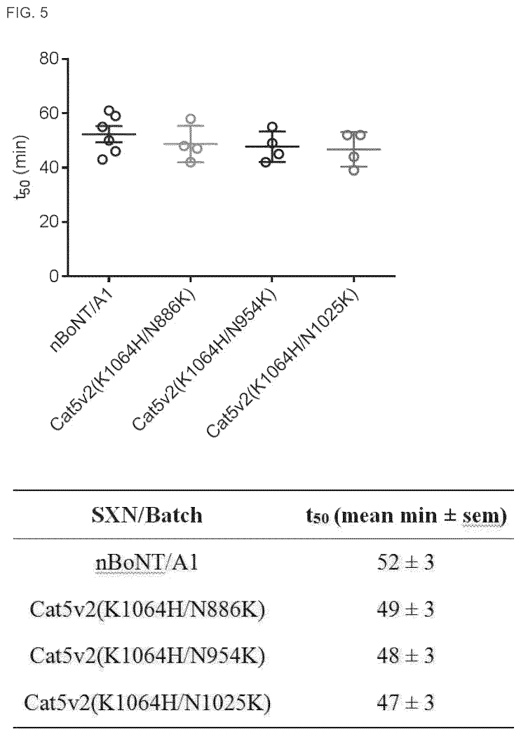

Potency of the modified BoNTs was further assessed using the mouse phrenic nerve hemi-diaphragm assay (mPNHD). FIG. 5 shows that the modified BoNTs retained the same ability to inhibit the contractile abilities of the mouse hemi-diaphragm as native BoNT/A1.

The in vivo mouse Digital Abduction Score (DAS) assay was used to assess potency as well as safety relative to native BoNT/A1. Both molecules (Cat-A [SEQ ID NO: 4] and Cat-B [SEQ ID NO: 6]) displayed a higher safety ratio relative to native BoNT/A1 and were slightly more potent. These data are presented in Table 3 below:

TABLE 3
DAS assay and safety ratio.

| | DAS | Dose | Dose for −10% | |
|---|---|---|---|---|
| | ED₅₀ | DAS 4 | ΔBW | Safety |
| Molecule | (pg/mouse) | (pg/mouse) | (pg/mouse) | Ratio |
| Native | 2 | 10-20 | 9.9-14.5 | 7 |
| BoNT/A1 (n = 5) | | | | |
| (SEQ ID NO: 2) | | | | |
| Modified, | 1.16 | 10-20 | 27.4 | 24 |
| “Cat-A” | | | | |
| (SEQ ID NO: 4) | | | | |
| Modified, | 1.79 | 25 | 47.6 | 27 |
| “Cat-B” | | | | |
| (SEQ ID NO: 6) | | | | |

DAS ED₅₀: Calculated dose inducing a DAS 2
Dose DAS 4: Experimental dose inducing a DAS 4
BW: Body weight
Dose for −10% ΔBW: Calculated dose inducing a decrease of 10% on BW in comparison to BW at D 0
Safety Ratio: Dose for −10% ΔBW / DAS ED₅₀

The Safety Ratio is a measure of a negative effect of BoNT treatment (weight loss) with respect to potency (half maximal digital abduction score (DAS)). It is calculated as the ratio between −10% Body Weight (BW) and the DAS ED₅₀, where −10% BW refers to the amount of BoNT (pg/animal) required for a 10% decrease in body weight, and ED₅₀ refers to the amount of BoNT (pg/animal) that will produce a DAS of 2.

The DAS assay is performed by injection of 20 μl of modified BoNT/A, formulated in Gelatin Phosphate Buffer, into the mouse gastrocnemius/soleus complex, followed by assessment of Digit Abduction as previously reported by Aoki (Aoki K R, Toxicon 39:1815-1820; 2001).

### Example 3

### Cloning, Expression and Purification of BoNT/AB Chimeras

BoNT/AB chimeric constructs 1, 2, 3A, 3B, and 3C (SEQ ID NO: 11 to 15, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 4.

TABLE 4
chimeric BoNT/AB constructs

| | SEQ | |
|---|---|---|
| | ID | |
| Molecule | NO | Sequence |
| Chimera 1 | 11 | A1:1-871 + B1:858-1291 |
| | | (E1191M/S1199Y) + His₁₀-tag |
| Chimera 2 | 12 | A1:1-874 + |
| | | ELGGGGSEL + |
| | | B1:858-1291 |
| | | (E1191M/S1199Y) + His₁₀-tag |
| Chimera 3A | 13 | A1:1-872 + B1:860-1291 |
| | | (E1191M/S1199Y) + His₁₀-tag |
| Chimera 3B | 14 | A1:1-872 + B1: 860-1291 |
| | | (E1191M/S1199Y) |
| Chimera 3C | 15 | A1:1-872 + B1: 860-1291 |

### Example 4

### Comparison of BoNT/AB Chimera 1, 2 and 3A

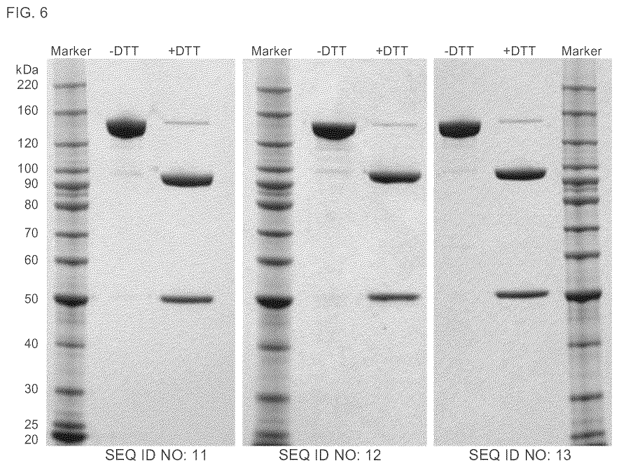

BoNT/AB chimera 1, 2 and 3A which have a C-terminal His₁₀ tag and E1191M/S1199Y double mutation were purified as described in Example 3 (FIG. 6) and tested for functional activity.

### Rat Spinal Cord Neurons Snap-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 24±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

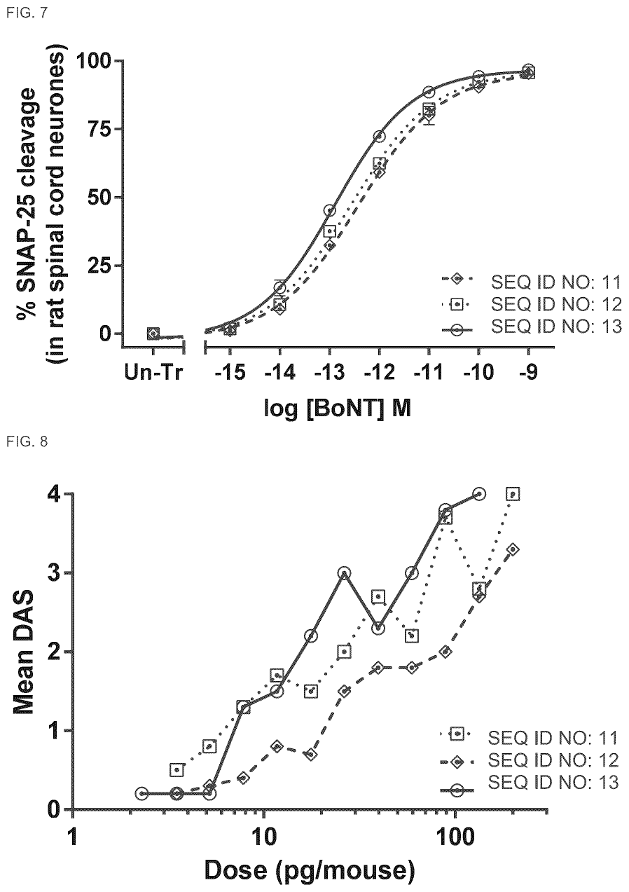

Table 5 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 7).

TABLE 5
pEC₅₀ values.

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

FIG. 8 shows the fitted curves for chimera 1, 2 and 3A (SEQ ID NO: 11, 12 and 13 respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 6) that provides the values for the calculated ED50 and the dose leading to DAS 4 (highest score) for each chimera.

Table 6 below provides the ED₅₀ and DAS 4 doses determined for unmodified recombinant BONT/A1 (rBoNT/A1—SEQ ID NO: 2) and chimeras 1, 2 and 3A in the mouse DAS assay. These results show that of the three chimeras, chimera 3A has the highest in vivo potency in inducing muscle weakening. Studies shown in FIG. 8 and Table 6 were performed in mice obtained from Charles River laboratories.

TABLE 6
ED₅₀ values.

| | ED₅₀ | DAS 4 dose |
|---|---|---|
| | (pg/mouse) | (pg/mouse) |
| rBoNT/A1 | 1 | 5 |
| Chimera 1 | 23 | 200 |
| Chimera 2 | 89 | >300 |
| Chimera 3A | 18 | 133 |

### Example 5

### Comparison of BoNT/AB Chimera 3B, 3C and Unmodified BoNT/A1

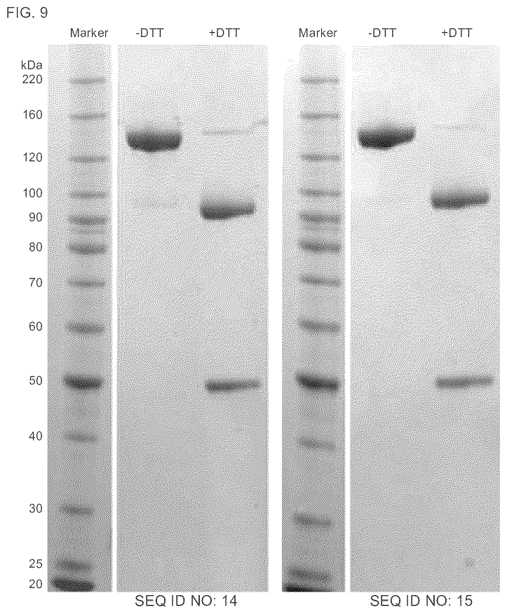

Untagged BoNT/AB chimera 3B and 3C, respectively with and without the presence of the E1191M/S1199Y double mutation (SEQ ID NO: 14 and 15) were purified as described in Example 3 (FIG. 9), and tested for functional activity using unmodified BoNT/A (SEQ ID NO: 2) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 mL tube. The cryovial was washed with 1 mL of Peri.4U® thawing medium supplied by the manufacturer and the medium was transferred drop-wise to the cell suspension to the 50 ml tube, prior to adding a further 2 mL of Peri.4U® thawing medium drop-wise to the 50 mL tube. Cells were then counted using a hemocytometer. After this, a further 6 mL of Peri.4UR thawing medium was added to the cell suspension. A cell pellet was obtained by centrifugation at 260×g (e.g. 1,100 RPM) for 6 minutes at room temperature. Cells were then resuspended in complete Peri.4UR culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4UR culture medium. The medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 48±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit lgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

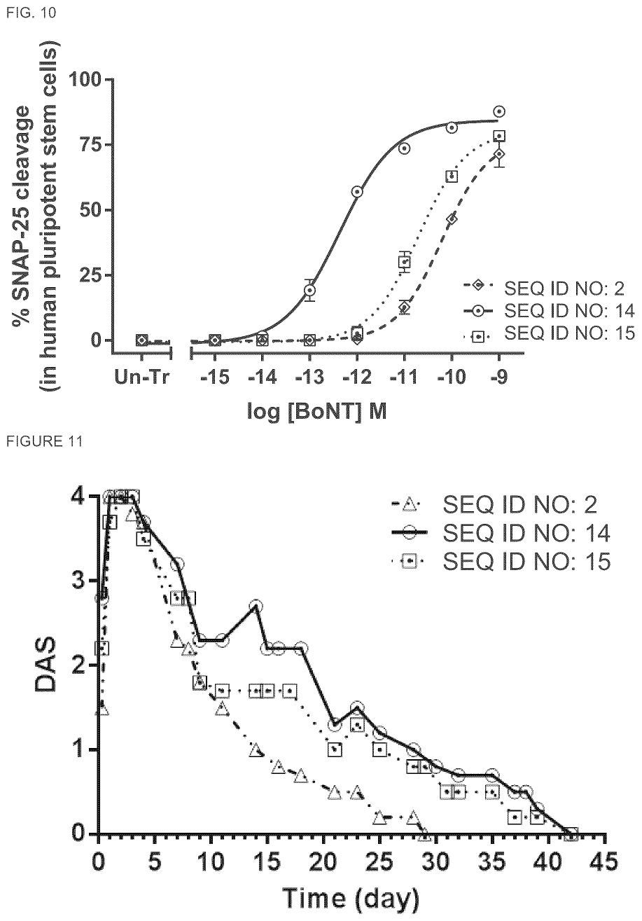

FIG. 10 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 10, Table 7).

TABLE 7
pEC₅₀ values.

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (DAS) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (D0) and thereafter once daily throughout the duration of the study. The average body weight, its standard deviation, and the standard error mean were calculated daily for each dose-group. To obtain the safety ratio for a BoNT (−10% ΔBW/ED₅₀), the dose at which at any time during the study the average weight of a dose-group was lower than 10% of the average weight at D0 of that same dose-group was divided by the ED₅₀ for the BoNT studied. The lethal dose was defined as the dose at which one or more of the animals within that dose-group died.

FIG. 11 shows the duration of muscle weakening over time in the mouse digit abduction scoring assay for unmodified BoNT/A, chimera 3B and chimera 3C (SEQ ID NO: 2, 14 and 15), showing that the chimera has longer duration of action.

Table 8 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ABW/ED₅₀), as defined in the text above. In comparison to rBoNT/A1, chimeras 3B and 3C have longer duration of action, a better safety ratio, and a higher lethal dose. Studies shown in FIG. 11 and Table 8 were performed in mice obtained from Janvier laboratories.

TABLE 8
DAS and Safety Ratios of the BoNT/AB chimeras.

| | | | Total | | |
|---|---|---|---|---|---|
| | | | duration of | | |
| | ED₅₀ | | action (day) | Mouse | |
| | (DAS 2) | DAS 4 | with lowest | lethal | Safety ratio |
| | Dose | dose | DAS 4 | dose | (−10% ΔBW/ |
| | (pg/mouse) | (pg/mouse) | dose | (pg) | ED₅₀) |
| rBoNT/A1 | 0.9 | 2.3 | 29 | 18 | 4.5 |
| Chimera 3B | 8.0 | 89 | 42 | 200 | 14.1 |
| Chimera 3C | 5.0 | 26 | 42 | 8.9 | 7.4 |

### Example 6

### Pre-Clinical Testing of Modified BoNT/A (SEQ ID NO: 4)

The modified BoNT/A “Cat-A” (SEQ ID NO: 4) was subjected to additional pre-clinical testing.

### Materials & Methods

### Rat Digit Abduction Score (DAS) Assay

To assess the effects of modified BoNT/A (SEQ ID NO: 4) on in vivo muscular activity, dose-response studies were conducted using the rat DAS assay. The rat DAS assay is based on the toe spreading reflex, a characteristic startle response, when the animal is briefly grasped. Following a single neurotoxin injection into the left peroneus muscle complex, the muscular weakness results in a reduction in digit abduction. The varying degrees of digit abduction are scored on a 5-point scale: 0=normal to 4=maximal reduction in digit abduction and leg extension (Broide R S, Rubino J, Nicholson G S, et al. The rat Digit Abduction Score (DAS) assay: A physiological model for assessing botulinum neurotoxin-induced skeletal muscle paralysis. Toxicon 2013; 71:18-24). DAS values were measured for the first five consecutive days after toxin injection and after this at intervals of two to three days until complete disappearance of the effect of modified BoNT/A (SEQ ID NO: 4) on the toe spreading reflex for lower doses and until recovery to DAS2 for doses resulting in DAS4. Transient BoNT-induced dose-dependent effects on body weight gain are considered evidence of a generalised toxin effect (Torii Y, Goto Y, Nakahira S, et al. Comparison of Systemic Toxicity between Botulinum Toxin Subtypes A1 and A2 in Mice and Rats. Basic Clin. Pharmacol. Toxicol. 2015; 116:524-528). At each evaluation time point rats were consequently weighed and side effects were noted. Dosing solutions of BoNT were masked (assigned random letters) before injection and until the end of the study. Potency was determined as the dose required to induce 50% of the effect (ED₅₀: dose leading to a DAS value of 2). To determine ED₅₀ and the 95% confidence intervals (CIs), doses ranging between 2.5 and 750 pg/kg were tested. Higher doses of 1, 1.5, 2, 2.4, 3, 4 and 5 ng/kg were also administered to assess possible side effects.

To evaluate the duration of action of modified BoNT/A (SEQ ID NO: 4) and compare it to the duration of action of unmodified BoNT/A (SEQ ID NO: 2), the median time necessary to return to a DAS2 reading of 2 was evaluated for the highest tolerated dose (no impact on body weight evolution compared to untreated rats) for both toxins in two independent, direct head-to-head studies.

### Rat Single Dose Studies

Rats received a single intramuscular (i.m.) injection of modified BoNT/A (SEQ ID NO: 4) at doses of 0, 0.1, 1 and 3 ng/kg administered into the right gastrocnemius muscle. Control animals received SEQ ID NO: 4 diluent in the right gastrocnemius. Animals were euthanised 7 days after treatment (ten males and ten females per group) or after a 13 or 26-week observation period (five males and five females per dose). Irwin test observations, for assessment of central nervous system function, were performed pretest (Day −1), on Day 8 and during Weeks 13 and 27. Other clinical (adverse) signs assessed for were limping, small toxin injected muscle size, and soft distended abdomen.

### Monkey Studies

Monkeys received single i.m. doses of 0, 0.1, 0.25 and 0.75 ng/kg modified BoNT/A (SEQ ID NO: 4) administered into the right gastrocnemius muscle. Animals were euthanised 7 days after treatment (three males and three females per group) or after a 13 or 26-week observation period (two males and two females per dose). Cardiovascular examinations, including haemodynamic, electrocardiogram and respiratory parameters, were performed by external telemetry pretest, on Days 8 and 15.

### Preliminary Enhanced EFD in Pregnant Rat

The objective of the study was to provide initial information on the effects of modified BoNT/A (SEQ ID NO: 4) on embryonic and foetal development of the rat when administered by the i.m. route throughout the period of organogenesis. Modified BoNT/A (SEQ ID NO: 4) was administered by daily i.m. injection (gastrocnemius) at dose levels of 0.02, 0.05 and 0.1 ng/kg/day to groups of nine mated female Sprague-Dawley rats from days 6 (G6) to 17 (G17) of gestation, inclusive. Clinical condition, body weight and food consumption were monitored throughout the study. The females were submitted to a caesarean examination on G21 and litter parameters were recorded. At necropsy, the females were examined macroscopically, the gravid uteri were weighed and for those who presented a small injected gastrocnemius muscle, this muscle and the contralateral muscle were weighed. All foetuses were weighed. The foetuses were then examined for external and visceral abnormalities and sexed. The heads of approximately half of the foetuses were fixed for internal examination by serial sectioning. The eviscerated carcasses of all fetuses were processed for skeletal examination.

### Preliminary Extended EFD in Pregnant Rabbit

The objective of the study was to provide initial information on the effects of modified BoNT/A (SEQ ID NO: 4) on embryonic and foetal development of the rabbit when administered by the i.m. route throughout the period of organogenesis. Modified BoNT/A (SEQ ID NO: 4) was administered by daily i.m. injection (gastrocnemius) at dose levels of 0.002, 0.005 and 0.01 ng/kg/day to groups of nine mated female New Zealand White rabbits from days 6 (G6) to 19 (G19) of gestation, inclusive. Clinical condition, body weight and food consumption were monitored throughout the study. The females were submitted to a caesarean examination on G29 and litter parameters were recorded. At necropsy, the females were examined macroscopically, the gravid uteri were weighed and for those who presented a small injected gastrocnemius muscle, this muscle and the contralateral muscle were weighed. All foetuses were weighed. The foetuses were then examined for external and visceral abnormalities and sexed. The heads of approximately half of the foetuses were fixed for internal examination by serial sectioning.

### Results

By carrying out the studies as indicated above, the following pharmacological data (indicated in Table 9 below) were obtained for a number of different species administered the modified BoNT/A.

TABLE 9
Pre-clinical results.

| Animal | Study Type | Results |
|---|---|---|
| Mouse | LD50 IP | 0.422 ng/kg |
| Rat | DAS | ED50 0.013 ng/kg |
| | | DAS4 0.125 pg/kg |
| | CMAP | 0.002 ng/kg: No spread |
| | Single Dose | 0.3 ng/kg: −25% |
| | Distant Spread | 0.8 ng/kg: −56% |
| | Single Dose | Estimated NOAEL 1.5 ng/kg |
| | | Estimated Lethal 3 ng/kg |
| Monkey | Single Dose | Estimated NOAEL 0.125 ng/kg |
| | | Lethal 0.375 ng/kg |
| Rat (Pregnant | pEFD | Maternal NOAEL and fetal |
| Female) | | NOEL 0.1 ng/kg/day |
| Rabbit | pEFD | Maternal NOAEL 0.005 ng/kg/day |
| (Pregnant | | Fetal NOEL 0.01 ng/kg/day |
| Female) | | |

Additionally, modified BoNT/A (SEQ ID NO: 4) was tested in a rat DAS assay to determine the duration of action when compared to Dysport®. Results are presented in Table 10 below:

TABLE 10
Duration of action.

| | Dysport ® | Modified BoNT/A |
|---|---|---|
| | 3 U/rat | 150 pg/rat |
| | 15 U/kg | 0.750 ng/kg |
| Duration of Action | 21.9 | 46.4 |
| (median days) | | |

These data show that the modified BoNT/A has a duration of action that is more than double that of Dysport®.

### Example 7

### Calculation of a Unit Dose of Modified BoNT/A (SEQ ID NO: 4) for Treating Limb Spasticity

In view of the pre-clinical pharmacology data obtained in Example 6 above, a suitable unit dose range (UD) for administration of modified BoNT/A in humans has been calculated. The studies show that modified BoNT/A provides a longer duration of action than unmodified BoNT/A while at the same time exhibiting an improved safety profile. This improved safety profile may be expressed by the high Safety Ratio described herein for the modified BoNT/A.

As modified BoNT/A shares the same mechanism of action as Dysport® (albeit with an increased Safety Ratio due to its modified properties), the lowest dose of modified BoNT/A for treating subjects with spasticity has been positioned for context relative to the labelled doses of Dysport® in that same muscle group:
In the Digit Abduction Score rat model, the ED₅₀ of modified BoNT/A is 13 pg/kg, and is more than 100-fold lower than the estimated no-observed-adverse-effect-level (NOAEL) of 1500 pg/kg in the same animal species. In the same rat model, the ED₅₀ of Dysport® is 0.5 U/kg. Based on these animal data, a dose of 2.6 ng of modified BoNT/A would estimate to a dose of 100 U Dysport® which is the lowest limit of the labelled range for treating a single spastic finger flexor muscle in an adult subject with upper limb spasticity.
The intraperitoneal mouse LD₅₀ was established at 8.44 pg. Under these conditions, a dose of 0.84 ng of modified BoNT/A corresponds to a dose of 100 U Dysport®.

The calculated lowest dose is thus 500 pg (0.5 ng). To provide some context and using the intraperitoneal mouse LD₅₀ data above, 0.5 ng modified BoNT/A equates to approximately 60 U Dysport®, and would thus be active when administered intramuscularly for treatment of limb spasticity.

The estimated NOAEL of 1.5 ng/kg of modified BoNT/A in rats corresponds to a 90 ng dose for a human of 60 kg body weight. In monkeys, the more sensitive of the two nonclinical species tested, the estimated NOAEL of 0.125 ng/kg of modified BoNT/A corresponds to a 7.5 ng dose for a human of 60 kg body weight.

The upper limit of the unit dose is thus calculated to be 7,500 pg (7.5 ng) as this remains below the rat NOAEL translated in human dose.

Thus, a suitable unit dose for treatment of limb spasticity using modified BoNT/A has been calculated at 500-7500 pg. Based on the pre-clinical data obtained, this is 59-889 Units of modified BoNT/A (and also corresponds to 59-889 Units of Dysport®) based on the calculated median lethal intraperitoneal dose (LD₅₀) in mice as determined using the mouse intraperitoneal Lethal Dose Assay.

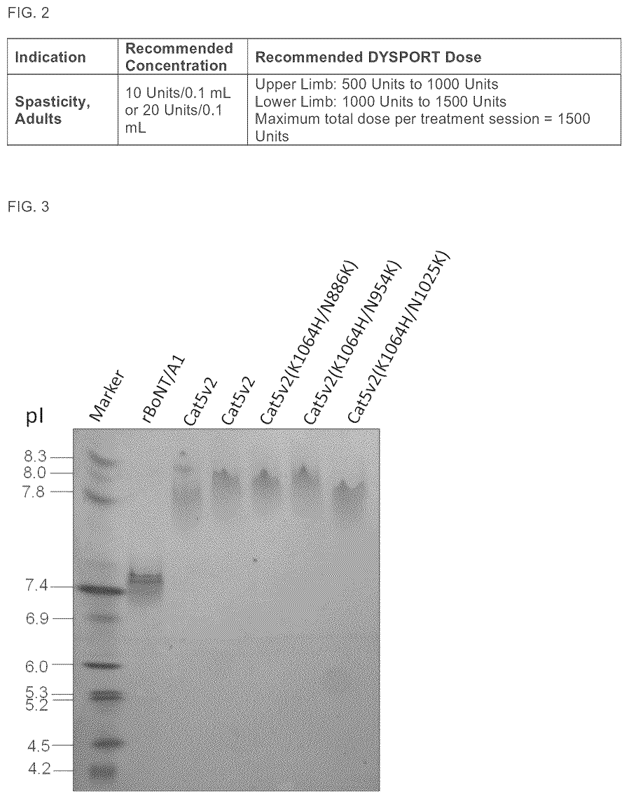

In view of the improved safety profile when compared to Dysport® as determined by the pre-clinical data of Example 6, total dosages (in units) administered in treating limb spasticity are expected to be almost 10× greater than that for Dysport®. The maximum total dose of Dysport® for treatment of upper and lower limb spasticity in adults is 1500 Units (see FIG. 2).

Advantageously, modified BoNT/A can be injected to a greater number of muscles in the treatment of limb spasticity before reaching the maximum dose. This is a significant and advantageous finding leading to improved treatment of limb spasticity while providing clinicians with a greater range of treatment options. For the first time, it also provides the option of being able to treat additional large muscles such as those of the shoulder, while also treating the elbow, forearm, and/or wrist well within the maximum dose.

### Example 8

### Dosage Regimen for Treating Adult Upper Limb Spasticity

Modified BoNT/A (e.g. SEQ ID NO:4) is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

The unit dose (UD) is 500-7,500 pg (59-889 Units).

Adult upper limb spasticity is treated by intramuscular injection according to the following dosage regimen (Table 11):

TABLE 11
Dosage regimen.

| | | | Total |
|---|---|---|---|
| Clinical Patterns | Muscles Injected | Dosage | Volume |
| Clenched fist | Flexor Digitorum | 1 × UD | 1 mL |
| | Superficialis (FDS) | | |
| | Flexor Digitorum | 1 × UD | 1 mL |
| | Profundus (FDP) | | |
| Flexed wrist | Flexor Carpi | 1 × UD | 1 mL |
| | Radialis (FCR) | | |
| | Flexor Carpi | 1 × UD | 1 mL |
| | Ulnaris (FCU) | | |
| Flexed elbow | Brachioradialis | 1 × UD | 1 mL |
| | Brachialis | 2 × UD | 2 mL |
| | Pronator Teres | 1 × UD | 1 mL |
| | Biceps Brachii | 2 × UD | 2 mL |
| | | 1 × or 2 × UD | 1-2 mL |
| Adducted/ | Triceps Brachii | 2 × UD | 2 mL |
| rotated | (long head) | | |
| shoulder | Pectoralis Major | 2 × UD | 2 mL |
| | Subscapularis | 2 × UD | 2 mL |
| | Latissimus Dorsi | 2 × UD | 2 mL |

A maximum total dosage administered is 15× UD. This corresponds to 112,500 pg/13,335 Units. This is more than 10× greater than the maximum total dosage of Dysport® that can be administered during treatment of adult upper limb spasticity without approaching toxic limits (a concern with conventional treatment regimens). Thus, the clinician is able to tailor treatment to the patient with the knowledge that 15× UD can be administered without any concern of toxicity, thereby allowing the treatment of additional muscles of the subject, including the shoulder, and/or ensuring each muscle receives a pharmaceutically effective dose.

### Example 9

### Dosage Regimen for Treating Adult Lower Limb Spasticity

Modified BoNT/A (e.g. SEQ ID NO:4) is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

The unit dose (UD) is 500-7,500 pg (59-889 Units).

Adult lower limb spasticity is treated by intramuscular injection according to the following dosage regimen (Table 12):

TABLE 12
Dosage regimen.

| Muscle Injected | Dosage |
|---|---|
| Gastrocnemius (Medial head) | 1 × UD |
| Gastrocnemius (Lateral head) | 1 × UD |
| Soleus | 3 × UD |
| Tibialis posterior | 2 × UD |
| Flexor digitorum longus | 1 × UD |
| Flexor hallucis longus | 1 × 2 × UD |

A maximum total dosage administered is 15× UD. This corresponds to 112,500 pg/13,335 Units. This is almost 10× greater than the maximum total dosage of Dysport® that can be administered during treatment of adult upper limb spasticity without approaching toxic limits (a concern with conventional treatment regimens). Thus, the clinician is able to tailor treatment to the patient with the knowledge that 15× UD can be administered without any concern of toxicity, thereby allowing the treatment of additional muscles of the subject and/or ensuring each muscle receives a pharmaceutically effective dose.

### Example 10

### Further Characterisation of a BoNT/AB Chimera (SEQ ID NO: 14)

BoNT/AB chimera SEQ ID NO: 14 was tested in a mouse LD₅₀ assay yielding a result of 1.202 ng/kg. 1 Unit of SEQ ID NO: 14 therefore corresponds to 24.04 pg in this assay.

Additionally, said BoNT/AB chimera was tested in a rat DAS assay to determine the duration of action (as per Example 6) when compared to Dysport®. Results are presented in Table 13 below:

TABLE 13
Duration of action.

| | Dysport ® | BoNT/AB |
|---|---|---|
| | 3 U/rat | 300 pg/rat |
| | 15 U/kg | 1.5 ng/kg |
| Duration of Action | 21.9 | 47.7 |
| (median days) | | |

In conclusion, the duration of action of BoNT/AB was much higher than Dysport® and similar to that of SEQ ID NO: 4. Thus, it is expected that the unit doses and dosage regimen for SEQ ID NO: 4 could similarly be applied to BoNT/AB to provide an improved treatment of limb spasticity.

### Example 11

### Calculation of a Unit Dose of Modified BoNT/A (SEQ ID NO: 14) for Treating Limb Spasticity

In view of pre-clinical pharmacology data, a suitable unit dose range (UD) for administration of modified BoNT/A in humans has been calculated.

A DAS ED₅₀ of 13 pg/kg was calculated for SEQ ID NO: 14. ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 14 in rats corresponds to a 0.8 ng dose for a human of 60 kg body weight.

Thus, the lower limit of a unit dose of 1000 pg was selected. An upper limit of the unit dose of 16,000 pg was selected, which is lower than the NOAEL of 4 ng/kg from both nonclinical safety species (rat and monkey) converted into human dose for 60 kg body weight.

In view of the improved safety profile the maximum total dose for the treatment of limb spasticity was set at 240,000 pg, which is derived from the NOAEL of 4 ng/kg from both nonclinical safety species (rat and monkey) converted into human dose for 60 kg body weight.

Advantageously, modified BoNT/A (SEQ ID NO: 14) can be injected to a greater number of muscles in the treatment of limb spasticity before reaching the maximum dose. This is a significant and advantageous finding leading to improved treatment of limb spasticity while providing clinicians with a greater range of treatment options. For the first time, it also provides the option of being able to treat additional large muscles such as those of the shoulder, while also treating the elbow, forearm, and/or wrist well within the maximum dose.

### Example 12

### Dosage Regimen for Treating Adult Upper Limb Spasticity Using a Modified BoNT/A (SEQ ID NO: 14)

Modified BoNT/A is provided as a lyophilised powder in a vial containing 36 ng of modified BoNT/A per vial. The lyophilised powder is reconstituted.

The unit dose (UD) is 1000-16,000 pg (42-666 Units [measured by mouse LD₅₀]).

Adult upper limb spasticity is treated by intramuscular injection according to the following dosage regimen (Table 14):

TABLE 14
Dosage regimen.

| | | | Total |
|---|---|---|---|
| Clinical Patterns | Muscles Injected | Dosage | Volume |
| Clenched fist | Flexor Digitorum | 1 × UD | 1 mL |
| | Superficialis (FDS) | | |
| | Flexor Digitorum | 1 × UD | 1 mL |
| | Profundus (FDP) | | |
| Flexed wrist | Flexor Carpi | 1 × UD | 1 mL |
| | Radialis (FCR) | | |
| | Flexor Carpi | 1 × UD | 1 mL |
| | Ulnaris (FCU) | | |
| Flexed elbow | Brachioradialis | 1 × UD | 1 mL |
| | Brachialis | 2 × UD | 2 mL |
| | Pronator Teres | 1 × UD | 1 mL |
| | Biceps Brachii | 2 × UD | 2 mL |
| | | 1 × or 2 × UD | 1-2 mL |
| Adducted/ | Triceps Brachii | 2 × UD | 2 mL |
| rotated | (long head) | | |
| shoulder | Pectoralis Major | 2 × UD | 2 mL |
| | Subscapularis | 2 × UD | 2 mL |
| | Latissimus Dorsi | 2 × UD | 2 mL |

A maximum total dosage administered is 15× UD. This corresponds to 240,000 pg/9,990 Units. Thus, the clinician is able to tailor treatment to the patient with the knowledge that 15× UD can be administered without any concern of toxicity, thereby allowing the treatment of additional muscles of the subject, including the shoulder, and/or ensuring each muscle receives a pharmaceutically effective dose.

### Example 13

### Dosage Regimen for Treating Adult Lower Limb Spasticity Using a Modified BoNT/A (SEQ ID NO: 14)

Modified BoNT/A is provided as a lyophilised powder in a vial containing 36 ng of modified BoNT/A per vial. The lyophilised powder is reconstituted.

The unit dose (UD) is 1000-16,000 pg (42-666 Units).

Adult lower limb spasticity is treated by intramuscular injection according to the following dosage regimen (Table 15):

TABLE 15
Dosage regimen.

| Muscle Injected | Dosage |
|---|---|
| Gastrocnemius (Medial head) | 1 × UD |
| Gastrocnemius (Lateral head) | 1 × UD |
| Soleus | 3 × UD |
| Tibialis posterior | 2 × UD |
| Flexor digitorum longus | 1 × UD |
| Flexor hallucis longus | 1 × 2 × UD |

A maximum total dosage administered is 15× UD. This corresponds to 240,000 pg/9,990 Units. Thus, the clinician is able to tailor treatment to the patient with the knowledge that 15× UD can be administered without any concern of toxicity, thereby allowing the treatment of additional muscles of the subject and/or ensuring each muscle receives a pharmaceutically effective dose.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1-119. (canceled)

120. A method for treating limb spasticity, wherein the method comprises administering a modified botulinum neurotoxin A (BoNT/A) by intramuscular injection to a plurality of affected muscles of a subject, wherein the modified BoNT/A is administered by way of a unit dose at the plurality of affected muscles,
wherein the plurality of affected muscles are selected from: a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered at an affected first group muscle and/or multiple unit doses are administered at an affected second group muscle, and wherein the modified BoNT/A comprises a BoNT/A light-chain and translocation domain, and a BoNT/B receptor binding domain (H_{C} domain).

121-122. (canceled)

123. The method of claim 120, wherein the limb spasticity is upper limb spasticity.

124-129. (canceled)

130. The method of claim 120, wherein the unit dose is 750 pg to 17,000 pg of modified BoNT/A.

131. The method of claim 120, wherein the total dose administered is 12,750 pg to 255,000 pg.

132-135. (canceled)

136. The method of claim 120, wherein the modified BoNT/A comprises a polypeptide sequence having at least 70% sequence identity to SEQ ID NO: 14.

137. A unit dosage form of modified botulinum neurotoxin A (BoNT/A), the unit dosage form comprising:
a. 31 Units to 707 Units of modified BoNT/A, wherein 1 Unit is an amount of the modified BoNT/A that corresponds to the calculated median lethal dose (LD₅₀) in mice; or
b. 750 pg to 17,000 pg of modified botulinum neurotoxin A (BoNT/A); and
c. a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

138. The unit dosage form according to claim 137, wherein the modified BoNT/A is:
a. 42 Units to 666 Units; or
b. 1000 pg to 16,000 pg.

139. A kit comprising:
a. the unit dosage form according to claim 137; and
b. instructions for use of the same in treating limb spasticity; and
c. a diluent.

140. A method for treating paediatric limb spasticity, wherein the method comprises administering a modified botulinum neurotoxin A (BoNT/A) by intramuscular injection to a plurality of affected muscles of a subject, wherein the modified BoNT/A is administered by way of a unit dose at the plurality of affected muscles,
wherein the plurality of affected muscles are selected from: a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
wherein a single unit dose is administered at an affected first group muscle and/or multiple unit doses are administered at an affected second group muscle.

141-177. (canceled)

178. The method of claim 140, wherein the unit dose is 225 pg to 8,000 pg.

179. The method of claim 178, wherein the total dose administered is 750 pg to 127,500 pg.

180. The method of claim 179, wherein the total dose administered is up to 120,000 pg.

181. The method of claim 180, wherein the total dose administered is 7,500 pg to 120,000 pg.

182-195. (canceled)

196. The method of claim 136, wherein the modified BoNT/A is in a di-chain form.

197. The unit dosage form according to claim 137, wherein the modified BoNT/A comprises a polypeptide sequence having at least 70% sequence identity to SEQ ID NO: 14.

198. The unit dosage form according to claim 197, wherein the modified BoNT/A is in a di-chain form.

199. The method of claim 120, wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277.
